(19) United States
(12) Patent Application Publication
(10) Pub. No.: US 2022/0202963 A1
(43) Pub. Date: Jun. 30, 2022

(54) RADIOLABELED COMPOUNDS

(71) Applicants: Hoffmann-La Roche Inc., Little Falls, NJ (US); ETH ZUERICH, Zuerich (CH)

(72) Inventors: Ludovic COLLIN, Huningue (FR); Martin EDELMANN, Lorrach (DE); Luca GOBBI, Buus (CH); Uwe GRETHER, Efringen-Kirchen (DE); Thomas HARTUNG, Lorrach (DE); Yingfang HE, Zurich (CH); Michael HONER, Zurich (CH); Benoit HORNSPERGER, Altkirch (FR); Carsten KROLL, Basel (CH); Linjing MU, Lenzburg (CH); Dieter MURI, Basel (CH); Fionn O'HARA, Basel (CH); Hans RICHTER, Grenzach-Wyhlen (DE); Martin RITTER, Mumpf (CH)

(73) Assignees: Hoffmann-La Roche Inc., Little Falls, NJ (US); ETH ZUERICH, Zuerich (CH)

(21) Appl. No.: 17/692,632

(22) Filed: Mar. 11, 2022

Related U.S. Application Data
(63) Continuation of application No. PCT/EP2020/075259, filed on Sep. 10, 2020.

(30) Foreign Application Priority Data
Sep. 12, 2019 (EP) 19196878.3

(51) Int. Cl. A61K 51/04; A61B 6/03

(52) U.S. Cl. CPC A61K 51/0463; A61B 6/037

(57) ABSTRACT

The invention provides new radiolabeled monoacylglycerol lipase (MAGL) inhibitors that are useful for medical imaging, such as positron-emission tomography (PET), single-photon emission computed tomography (SPECT) and/or autoradiography.

### CROSS REFERENCE TO RELATED APPLICATIONS

This application is a continuation of International Application No. PCT/EP2020/075259, filed on Sep. 10, 2020, which claims the benefit of EP Application No. 19196878.3, filed on Sep. 12, 2019, the disclosures of which are incorporated herein by reference in their entireties.

### FIELD OF THE INVENTION

The present invention relates to radiolabeled organic compounds. More particularly, the present invention relates to radiolabeled monoacylglycerol lipase (MAGL) inhibitors that are useful for medical imaging, such as positron-emission tomography (PET), single-photon emission computed tomography (SPECT) and/or autoradiography.

### BACKGROUND OF THE INVENTION

It has been found that the radiolabeled compounds described herein may be used for the molecular imaging of monoacylglycerol lipase (MAGL). Molecular imaging is based on the selective and specific interaction of a molecular probe (e.g. a radiotracer) with a biological target (for instance a receptor, an enzyme, an ion channel, a misfolded protein or any other cellular or extracellular component that is able to bind or retain the molecular probe) which is visualized through PET, nuclear magnetic resonance, near infrared or other methods. PET, a nuclear medical imaging modality, is ideally suited to produce three-dimensional images that provide important information on the distribution of a biological target in a given organ, or on the metabolic activity of such organ or cell or on the ability of a drug to enter such organ, bind to a biological target and/or modify biological processes. Since PET is a non-invasive imaging technique it can be used to investigate the pathophysiology of a disease and the action of a drug on a given molecular target or cellular processes in humans and in animals. The availability of a PET radiotracer specific for a given molecular target can facilitate diagnosis and monitoring of progression of a disease by demonstrating and quantifying pathophysiological changes taking place as a consequence of the disease. In addition, a PET radiotracer may facilitate drug development by supporting patient stratification and the understanding of the mechanism of action of a drug.

The human brain is a complex organ, consisting of millions of intercommunicating neurons. The understanding of abnormalities relating to diseases is the key to the future development of effective diagnosis and novel therapeutics. The study of biochemical abnormalities in human is rapidly becoming an essential and integral component of the drug discovery and development process. Over recent years, there has been a growing use of human medical imaging to assess pathologies, disease processes and drug action. These imaging modalities include PET, MRI, CT, ultrasound, EEG, SPECT and others (British Medical Bulletin, 2003, 65, 169-177). Therefore, the use of non-invasive imaging modalities, e.g. PET, is an invaluable tool for the development of drugs in the future. Non-invasive nuclear imaging techniques can be used to obtain basic and diagnostic information about the physiology and biochemistry of a variety of living subjects. These techniques rely on the use of sophisticated imaging instrumentation that is capable of detecting radiation emitted from radiotracers administered to such living subjects. The information obtained can be reconstructed to provide planar and tomographic images that reveal distribution of the radiotracer as a function of time. The use of radiotracers can result in images which contain information on the structure, function and most importantly, the physiology and biochemistry of the subject. Much of this information cannot be obtained by other means. The radiotracers used in these studies are designed to have defined behaviors in vivo which permit the determination of specific information concerning the physiology or biochemistry of the subject. Currently, radiotracers are available for obtaining useful information concerning cardiac function, myocardial blood flow, lung perfusion, liver function, brain blood flow, regional brain glucose and oxygen metabolism, function of several brain receptors and enzymes and visualization of amyloid beta plaques and tau deposits in Alzheimer's disease (PET Molecular Imaging and Its Biological Applications, Eds. Michael E. Phelps, Springer, New York, 2004. Ametamy S. et al., Chem. Rev., 2008, 108, 1501-1516. Nordberg A. et al. Nat. Rev. Neurol., 2010, 6, 78-87).

Furthermore, PET imaging provides a non-invasive and quantitative assay of normal and abnormal neurochemistry in human at an early stage of the drug development to enhance the efficient and effective discovery of therapeutics. Understanding disease mechanisms in human using non-invasive techniques is intimately connected with future developments in the diagnosis and management of diseases and of novel therapeutics. Tracer doses of labeled compounds enable the early evaluation of novel drugs, e.g. by bio-distribution studies or by receptor occupancy studies to optimize drug-dosing regime and characterizing downstream responses of drug action.

The radionuclides commonly used in PET include ¹¹C, ¹³N, ¹⁵O or ¹⁸F. In principle, it is possible to label all drugs by replacing one of the parent compound atoms with a PET nuclide, but only a few are found applicable as imaging agents in vivo in humans. The radioactive half-time of ¹¹C, ¹³N, ¹⁵O and ¹⁸F are 20, 10, 2 and 110 min, respectively. These short half-lives endow a number of advantages to their use as tracers to probe biological processes in vivo using PET. Repeat studies in the same subject within the same day are made possible.

Tritium labeled compounds are particularly valuable and widely used for studies involving high resolution autoradiography. The physical (nuclear) properties of tritium, the low maximum beta energy (18 keV) of the radiation and the high maximum specific activity (29 Ci/mg atom of hydrogen), makes tritium the ideal isotope for determining the precise localization of compounds, drugs and hormones for example, in biological specimens.

The present radiolabeled compounds are MAGL inhibitors. Suppressing the action and/or the activation of MAGL is a promising new therapeutic strategy for the treatment or prevention of neuroinflammation, neurodegenerative diseases, pain, cancer and mental disorders. Furthermore, suppressing the action and/or the activation of MAGL is a promising new therapeutic strategy for providing neuroprotection and myelin regeneration.

Several PET tracers have been reported for the selective imaging of MAGL, yet most of these are based on covalent inhibitor structures. Covalent and irreversible binding of a radiotracer is associated with difficulties in the quantification of the signal by kinetic modeling, and is thus considered to be an unwanted attribute for a radiotracer. Examples of such tracers include [¹¹C]SAR12303 (T. Yamasaki et al., NeuroImage 176 (2018) 313-320), [¹¹C]MA-PB-1 (A. Muneer et al., European Journal of Medicinal Chemistry 136 (2017) 104-113), 1,1,1,3,3,3-hexafluoropropan-2-yl-3-(1-benzyl-1H-pyrazol-3-yl)azetidine-1-[¹¹C]carboxylate (W. Mori et al., Bioorganic & Medicinal Chemistry 27 (2019) 3568-3573) or compounds described in a patent application by Abide (WO2017/143283 A1).

[¹⁸F]T-401 represents an exceptional case of a non-covalent, reversible PET tracer targeting MAGL (Y. Hattori et al., J. Med. Chem. 62 (2019), 2362-2375).

In conclusion, there continues to be a need for alternative, non-covalent, reversible PET tracers to validate target engagement of therapeutic MAGL inhibitors, as well as to investigate MAGL levels under normal and disease conditions.

### SUMMARY OF THE INVENTION

In a first aspect, the present invention provides a radiolabeled compound selected from the group consisting of:
(4aR,8aS)-6-[3-[2-[2-fluoro-6-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[3-[2-(2-fluoro-4-methyl-phenyl)ethyl]azetidine-1-carbonyl]-4,4a,5,7,8,8a-hexahydropyrido[4,3-b][1,4]oxazin-3-one;
(4aR,8aS)-6-[3-[2-[4-methoxy-2-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[3-(4-cyclobutylphenyl)azetidine-1-carbonyl]-4,4a,5,7,8,8a-hexahydropyrido[4,3-b][1,4]oxazin-3-one;
(4aR,8aS)-6-[6-[(2,6-difluorophenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[6-[(2-methoxyphenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[3-[4-(cyclopentoxy)phenyl]azetidine-1-carbonyl]-4,4a,5,7,8,8a-hexahydropyrido[4,3-b][1,4]oxazin-3-one;
(4aR,8aS)-6-[3-(4-isobutoxyphenyl)azetidine-1-carbonyl]-4,4a,5,7,8,8a-hexahydropyrido[4,3-b][1,4]oxazin-3-one;
(4aR,8aS)-6-[3-[(Z)-2-fluoro-2-(3-fluoro-4-methyl-phenyl)vinyl]azetidine-1-carbonyl]-4,4a,5,7,8,8a-hexahydropyrido[4,3-b][1,4]oxazin-3-one; and
(4aR,8aS)-6-[3-[(E)-2-fluoro-2-(3-fluoro-4-methyl-phenyl)vinyl]azetidine-1-carbonyl]-4,4a,5,7,8,8a-hexahydropyrido[4,3-b][1,4]oxazin-3-one;
or a pharmaceutically acceptable salt thereof, comprising one or more radioisotopes.

In a further aspect, the present invention provides a radiolabeled compound described herein for use in monoacylglycerol lipase (MAGL) occupancy studies.

In a further aspect, the present invention provides a radiolabeled compound described herein for use in diagnostic imaging of monoacylglycerol lipase (MAGL) in the brain of a mammal.

In a further aspect, the present invention provides a pharmaceutical composition comprising a radiolabeled compound described herein and a pharmaceutically acceptable carrier.

### BRIEF DESCRIPTION OF THE FIGURES

FIG. 1 shows the in vitro binding to MAGL of [³H]1, [³H]2, [³H]3 and [³H]4 in sagittal mouse brain sections demonstrating high selectivity for MAGL and low non-specific binding. Top row: wt animals. Bottom row: MAGL ko animals.

FIG. 2 shows the in vitro binding to MAGL of [¹¹C]1 (A) and [¹¹C]5 (B) to sagittal brain sections from Wistar rats. Selectivity and non-specific binding are assessed by co-incubation with a high concentration of a cold MAGL inhibitor.

FIG. 3 shows the in vivo binding of [³H]2 as assessed by ex vivo autoradiography in CD(SD) rats.

FIG. 4 shows the time activity curves of the whole brain from [¹¹C]1 in MAGL ko mice and corresponding WT.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition, these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The abbreviation “MAGL” refers to the enzyme monoacylglycerol lipase. The terms “MAGL” and “monoacylglycerol lipase” are used herein interchangeably.

The term “one or more” means from one substituent to the highest chemically possible number of substitution, i.e. replacement of one atom up to replacement of all atoms by their respective radioisotopes.

The term “mammal” includes humans, non-human primates such as chimpanzees and other apes and monkey species, farm animals such as cattle, horses, sheep, goats, and swine, domestic animals such as rabbits, dogs, and cats, laboratory animals including rodents, such as rats, mice, and guinea pigs. In certain embodiments, a mammal is a human. The term mammal does not denote a particular age or sex.

The terms “pharmaceutically acceptable excipient” and “therapeutically inert excipient” can be used interchangeably and denote any pharmaceutically acceptable ingredient in a pharmaceutical composition having no therapeutic activity and being non-toxic to the subject administered, such as disintegrators, binders, fillers, solvents, buffers, tonicity agents, stabilizers, antioxidants, surfactants, carriers, diluents or lubricants used in formulating pharmaceutical products.

### Imaging Isotopes and Imaging

Diagnostic techniques in nuclear medicine use radioactive tracers which emit gamma rays from within the body. These tracers are generally short-lived isotopes linked to chemical compounds which permit specific physiological processes to be scrutinized. They can be given by injection, inhalation or orally. The first type is where single photons are detected by a gamma camera which can view organs from many different angles. The camera builds up an image from the points from which radiation is emitted; this image is enhanced by a computer and viewed by a physician on a monitor for indications of abnormal conditions.

Positron Emission Tomography (PET) is a precise and sophisticated technique using isotopes produced in a cyclotron. A positron-emitting radionuclide is introduced, usually by injection, and accumulates in the target tissue. As it decays it emits a positron, which promptly combines with a nearby electron resulting in the simultaneous emission of two identifiable gamma rays in opposite directions. These are detected by a PET camera and give a very precise indication of their origin. PET's most important clinical role is in oncology, with fluorine-18 fluorodeoxyglucose ([¹⁸F]FDG) as the tracer, since it has proven to be the most accurate non-invasive method of detecting and evaluating most cancers. It is also well used in cardiac and brain imaging.

A number of medical diagnostic procedures, including PET and SPECT, utilize radiolabeled compounds and are well known in the art. PET and SPECT are very sensitive techniques and require small quantities of radiolabeled compounds, called tracers. The labeled compounds are transported, accumulated and converted in vivo in a similar manner as the corresponding non-radioactively labeled compound. Tracers, or probes, can be radiolabeled with a radionuclide useful for PET imaging, such as ¹¹C, ¹³N, ¹⁵O, ¹⁸F, ⁶⁴Cu, and ¹²⁴I, or with a radionuclide useful for SPECT imaging, such as ⁹⁹Tc, ⁷⁷Br, ⁶¹Cu, ¹⁵³Gd, ¹²³I, ¹²⁵I, ¹³¹I and ³²P. These are non-limiting examples of “radioisotopes” (also known as imaging isotopes) as that term is used herein.

PET creates images based on the distribution of molecular imaging tracers carrying positron-emitting isotopes in the tissue of the patient. The PET method has the potential to detect malfunction on a cellular level in the investigated tissues or organs. PET has been used in clinical oncology, such as for the imaging of tumors and metastases, and has been used for diagnosis of certain brain diseases, as well as mapping brain and heart function. Similarly, SPECT can be used to complement any gamma imaging study, where a true 3D representation can be helpful, for example, imaging tumor, infection (leukocyte), thyroid or bones.

Regarding radiohalogens, ¹²⁵I isotopes are useful for laboratory testing but they will generally not be useful for diagnostic purposes because of the relatively long half-life (60 days) and low gamma-emission (30-65 keV) of ¹²⁵I. The isotope ¹²³I has a half-life of thirteen hours and gamma energy of 159 keV, and it is therefore typical that labeling of ligands to be used for diagnostic purposes would be with this isotope or with ¹⁸F (half-life of 2 hours). Other imaging isotopes which may be used include ¹³¹I, ⁷⁷Br and ⁷⁶Br.

In another embodiment, compounds of the present invention contain a radioactive isotope of carbon as the radiolabel. This refers to a compound that comprises one or more radioactive carbon atoms, preferably ¹¹C, with a specific activity above that of the background level for that atom. It is well known that naturally occurring elements are present in the form of varying isotopes, some of which are radioactive. The radioactivity of the naturally occurring elements is a result of the natural distribution or abundance of these isotopes, and is commonly referred to as a background level. The carbon labeled compounds of the present invention have a specific activity that is higher than the natural abundance, and therefore above the background level. The carbon labeled compositions of the present invention can be used for tracing, imaging, radiotherapy, and the like.

Those skilled in the art are familiar with the various ways to detect labeled compounds for imaging purposes. For example, positron emission tomography (PET) or single photon emission computed tomography (SPECT) can be used to detect radiolabeled compounds. The label that is introduced into the compound can depend on the detection method desired. Those skilled in the art are familiar with PET detection of a positron-emitting atom, such as ¹⁸F. The present invention is also directed to specific compounds described herein where the ¹⁸F atom is replaced with a non-radiolabeled fluorine atom. Those skilled in the art are familiar with SPECT detection of a photon-emitting atom, such as ¹²³I or ⁹⁹Tc.

The radioactive diagnostic or detection agent should have sufficient radioactivity and radioactivity concentration which can assure reliable diagnosis and detection. The desired level of radioactivity can be attained by the methods provided herein for preparing compounds.

Typically, a prerequisite for an in vivo imaging agent of the brain is the ability to cross the intact blood-brain barrier. In a first step of a method of imaging, a labeled compound is introduced into a tissue or a patient in a detectable quantity. The compound is typically part of a pharmaceutical composition and is administered to the tissue or the patient by methods well known to those skilled in the art. Typically, administration is intravenously.

In other embodiments of the invention, the labeled compound is introduced into a patient in a detectable quantity and after sufficient time has passed for the compound to become associated with MAGL, the labeled compound is detected noninvasively. In another embodiment of the invention, a labeled compound is introduced into a patient, sufficient time is allowed for the compound to become associated with MAGL, and then a sample of tissue from the patient is removed and the labeled compound in the tissue is detected apart from the patient. In another embodiment of the invention, a tissue sample is removed from a patient and a labeled compound is introduced into the tissue sample. After a sufficient amount of time for the compound to become bound to MAGL, the compound is detected.

A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The amount of time necessary can easily be determined by introducing a detectable amount of a labeled compound into a patient and then detecting the labeled compound at various times after administration.

The administration of the labeled compound to a patient can be by a general or local administration route. For example, the labeled compound may be administered to the patient such that it is delivered throughout the body. Alternatively, the labeled compound can be administered to a specific organ or tissue of interest. For example, it is desirable to locate and quantitate MAGL protein levels in the brain in order to diagnose or track the progress of e.g., neuroinflammation in a patient.

One or more imaging isotopes can be incorporated into the MAGL inhibitors disclosed herein by replacing one or more atoms (e.g., hydrogen or carbon atoms) in the MAGL inhibitors with an imaging isotope. The incorporation of an imaging isotope can be carried out using known techniques. For example, techniques may be based on nucleophilic or electrophilic ¹¹F-fluorination of suitable precursors as reviewed, for example, in Medicinal Chemistry Approaches to Personalized Medicine (Lackey, Roth Eds), Chapter 12 (Wiley-VCH, ISBN 978-3-527-33394-3). See also U.S. Patent Application No. 2011/0182812, incorporated herein by reference in its entirety. Furthermore, several methods exist for incorporating ¹¹C (Peter J. H. Scott, Angew. Chem. Int. Ed. 2009, 48, 6001-6004) or ¹⁸F (Sean Preshlock et al., Chem. Rev. 2016, 116, 719-766. Frederic Dollé (2008) Fluorine-18 chemistry for molecular imaging with positron emission tomography. In Fluorine and Health: Molecular Imaging, Biomedical Materials and Pharmaceuticals (Tressaud, A. and Haufe, G., eds), pp. 3-66, Elsevier) into compounds.

Radiolabeled compounds have structures depicted by the formulae given herein except that one or more atoms are replaced by an atom having a selected atomic mass or mass number. Examples of radiolabels that can be incorporated into compounds of the invention include isotopes of hydrogen, carbon, nitrogen, oxygen, phosphorous, fluorine, chlorine, and iodine, such as ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Substitution with heavier isotopes such as deuterium (i.e., ²H) may afford certain therapeutic advantages resulting from greater metabolic stability, for example increased in vivo half-life or reduced dosage requirements. Substitution with ²H may in particular be used to prevent the formation of undesired radiometabolites or to block radiodefluorination.

### Compounds of the Invention

In a first aspect (A1), the present invention provides a radiolabeled compound selected from the group consisting of
(4aR,8aS)-6-[3-[2-[2-fluoro-6-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[3-[2-(2-fluoro-4-methyl-phenyl)ethyl]azetidine-1-carbonyl]-4,4a,5,7,8,8a-hexahydropyrido[4,3-b][1,4]oxazin-3-one;
(4aR,8aS)-6-[3-[2-[4-methoxy-2-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[3-(4-cyclobutylphenyl)azetidine-1-carbonyl]-4,4a,5,7,8,8a-hexahydropyrido[4,3-b][1,4]oxazin-3-one;
(4aR,8aS)-6-[6-[(2,6-difluorophenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[6-[(2-methoxyphenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[3-[4-(cyclopentoxy)phenyl]azetidine-1-carbonyl]-4,4a,5,7,8,8a-hexahydropyrido[4,3-b][1,4]oxazin-3-one;
(4aR,8aS)-6-[3-(4-isobutoxyphenyl)azetidine-1-carbonyl]-4,4a,5,7,8,8a-hexahydropyrido[4,3-b][1,4]oxazin-3-one;
(4aR,8aS)-6-[3-[(Z)-2-fluoro-2-(3-fluoro-4-methyl-phenyl)vinyl]azetidine-1-carbonyl]-4,4a,5,7,8,8a-hexahydropyrido[4,3-b][1,4]oxazin-3-one; and
(4aR,8aS)-6-[3-[(E)-2-fluoro-2-(3-fluoro-4-methyl-phenyl)vinyl]azetidine-1-carbonyl]-4,4a,5,7,8,8a-hexahydropyrido[4,3-b][1,4]oxazin-3-one;
or a pharmaceutically acceptable salt thereof, comprising one or more radioisotopes.

The invention also provides the following enumerated Embodiments (E) of the first aspect (A1) of the invention:
E1. The radiolabeled compound comprising one or more radioisotopes according to A1, selected from the group consisting of
   (4aR,8aS)-6-[3-[2-[2-fluoro-6-(trifluoromethyl)phenyl]ethyl]azetidine-1-carbonyl]-4,4a,5,7,8,8a-hexahydropyrido[4,3-b][1,4]oxazin-3-one;
   (4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one;
   (4aR,8aS)-6-[3-[2-(2-fluoro-4-methyl-phenyl)ethyl]azetidine-1-carbonyl]-4,4a,5,7,8,8a-hexahydropyrido[4,3-b][1,4]oxazin-3-one;
   (4aR,8aS)-6-[3-[2-[4-methoxy-2-(trifluoromethyl)phenyl]ethyl]azetidine-1-carbonyl]-4,4a,5,7,8,8a-hexahydropyrido[4,3-b][1,4]oxazin-3-one;
   (4aR,8aS)-6-[3-(4-cyclobutylphenyl)azetidine-1-carbonyl]-4,4a,5,7,8,8a-hexahydropyrido[4,3-b][1,4]oxazin-3-one; and
   (4aR,8aS)-6-[6-[(2,6-difluorophenyl)methyl]-2-azaspiro[3.3]heptane-2-carbonyl]-4,4a,5,7,8,8a-hexahydropyrido[4,3-b][1,4]oxazin-3-one;
   or a pharmaceutically acceptable salt thereof.
E2. The radiolabeled compound comprising one or more radioisotopes according to A1, selected from the group consisting of
   (4aR,8aS)-6-[3-[2-[2-fluoro-6-(trifluoromethyl)phenyl]ethyl]azetidine-1-carbonyl]-4,4a,5,7,8,8a-hexahydropyrido[4,3-b][1,4]oxazin-3-one; and
   (4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one;
   or a pharmaceutically acceptable salt thereof.
E3. The radiolabeled compound according to any one of A1 and E1 to E2, or a pharmaceutically acceptable salt thereof, wherein said one or more radioisotopes are imaging isotopes for positron-emission tomography (PET), single-photon emission computed tomography (SPECT) and/or autoradiography.
E4. The radiolabeled compound according to any one of A1 and E1 to E3, or a pharmaceutically acceptable salt thereof, wherein said one or more radioisotopes are independently selected from the group consisting of ³H, ¹¹C, ¹⁴C, ¹³N, ¹⁵O, and ¹⁸F.
E5. The radiolabeled compound according to any one of A1 and E1 to E3, or a pharmaceutically acceptable salt thereof, wherein said one or more radioisotopes are independently selected from the group consisting of ³H, ¹¹C, and ¹⁸F.
E6. The radiolabeled compound according to any one of A1 and E1 to E3, or a pharmaceutically acceptable salt thereof, wherein said one or more radioisotopes are independently selected from the group consisting of ¹¹C and ¹⁸F.
E7. The radiolabeled compound according to any one of A1 and E1 to E6, or a pharmaceutically acceptable salt thereof, comprising 1-4 radioisotopes, e.g. 1, 2, 3 or 4 radioisotopes.
E8. The radiolabeled compound according to any one of A1 and E1 to E6, or a pharmaceutically acceptable salt thereof, comprising 1-3 radioisotopes, e.g. 1, 2 or 3 radioisotopes.
E9. The radiolabeled compound according to any one of A1 and E1 to E6, or a pharmaceutically acceptable salt thereof, comprising 1 radioisotope.
E10. The radiolabeled compound comprising one or more radioisotopes according to any one of A1 and E1 to E9, selected from the group consisting of

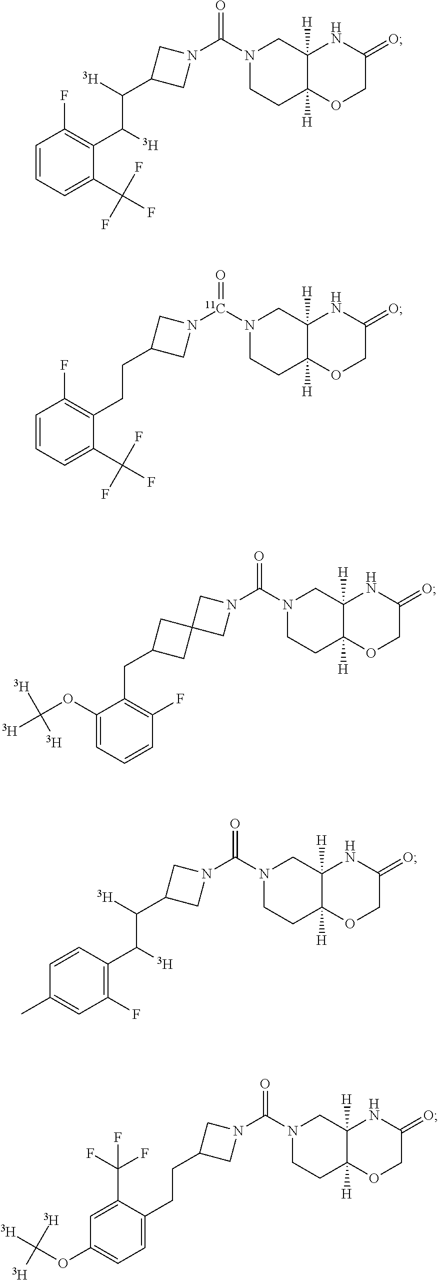 [SMILES: [3H]C(c1c(F)cccc1C(F)(F)F)C([3H])C1CN(C(=O)N2CC[C@@H]3OCC(=O)N[C@@H]3C2)C1 ; O=C1CO[C@H]2CCN([11C](=O)N3CC(CCc4c(F)cccc4C(F)(F)F)C3)C[C@H]2N1 ; [3H]C([3H])([3H])Oc1cccc(F)c1CC1CC2(C1)CN(C(=O)N1CC[C@@H]3OCC(=O)N[C@@H]3C1)C2 ; [3H]C(c1ccc(C)cc1F)C([3H])C1CN(C(=O)N2CC[C@@H]3OCC(=O)N[C@@H]3C2)C1 ; [3H]C([3H])([3H])Oc1ccc(CCC2CN(C(=O)N3CC[C@@H]4OCC(=O)N[C@@H]4C3)C2)c(C(F)(F)F)c1]

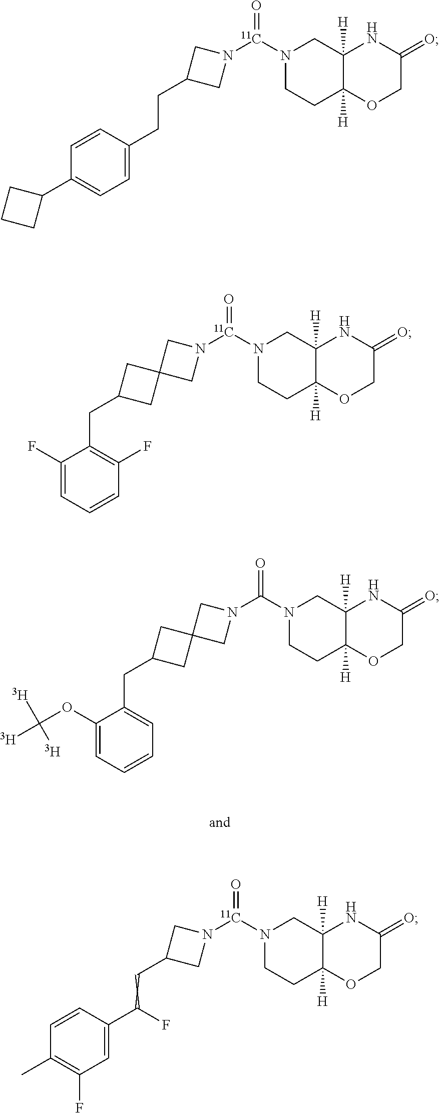 [SMILES: O=C1CO[C@H]2CCN([11C](=O)N3CC(CCc4ccc(C5CCC5)cc4)C3)C[C@H]2N1 ; O=C1CO[C@H]2CCN([11C](=O)N3CC4(CC(Cc5c(F)cccc5F)C4)C3)C[C@H]2N1 ; [3H]C([3H])([3H])Oc1ccccc1CC1CC2(C1)CN(C(=O)N1CC[C@@H]3OCC(=O)N[C@@H]3C1)C2 ; Cc1ccc(C(F)=CC2CN([11C](=O)N3CC[C@@H]4OCC(=O)N[C@@H]4C3)C2)cc1F]

or a pharmaceutically acceptable salt thereof.
E11. The radiolabeled compound comprising one or more radioisotopes according to any one of A1 and E1 to E9, selected from the group consisting of

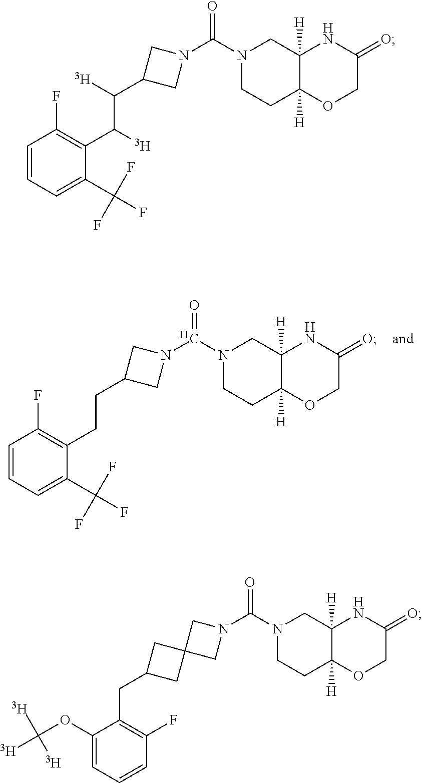 [SMILES: [3H]C(c1c(F)cccc1C(F)(F)F)C([3H])C1CN(C(=O)N2CC[C@@H]3OCC(=O)N[C@@H]3C2)C1 ; O=C1CO[C@H]2CCN([11C](=O)N3CC(CCc4c(F)cccc4C(F)(F)F)C3)C[C@H]2N1 ; [3H]C([3H])([3H])Oc1cccc(F)c1CC1CC2(C1)CN(C(=O)N1CC[C@@H]3OCC(=O)N[C@@H]3C1)C2]

or a pharmaceutically acceptable salt thereof.

### Using the Compounds of the Invention

The radiolabeled compounds of the present invention may be used for example as non-covalent, reversible PET tracers to validate target engagement of therapeutic MAGL inhibitors, as well as to investigate MAGL levels under normal and disease conditions.

Thus, in one aspect, the present invention provides the use of a radiolabeled compound disclosed herein for medical imaging, such as positron-emission tomography (PET), single-photon emission computed tomography (SPECT) and/or autoradiography.

In a further aspect, the present invention provides the radiolabeled compounds disclosed herein for use in medical imaging, such as positron-emission tomography (PET), single-photon emission computed tomography (SPECT) and/or autoradiography.

In a further aspect, the present invention provides a method of medical imaging, such as positron-emission tomography (PET), single-photon emission computed tomography (SPECT) and/or autoradiography, comprising contacting monoacylglycerol lipase (MAGL) with a radiolabeled compound disclosed herein.

In a further aspect, the present invention provides the use of a radiolabeled compound disclosed herein for the preparation of a medicament for medical imaging, such as positron-emission tomography (PET), single-photon emission computed tomography (SPECT) and/or autoradiography.

In a further aspect, the present invention provides the radiolabeled compounds disclosed herein for use in monoacylglycerol lipase (MAGL) occupancy studies.

In a further aspect, the present invention provides the use of the radiolabeled compounds disclosed herein for monoacylglycerol lipase (MAGL) occupancy studies.

In a further aspect, the present invention provides a method of studying monoacylglycerol lipase (MAGL) occupancy, comprising contacting MAGL with a radiolabeled compound disclosed herein.

In a further aspect, the present invention provides the use of the radiolabeled compounds disclosed herein for the preparation of a medicament for monoacylglycerol lipase (MAGL) occupancy studies.

In a further aspect, the present invention provides the radiolabeled compounds disclosed herein for use in diagnostic imaging of monoacylglycerol lipase (MAGL) in the brain of a mammal.

In a further aspect, the present invention provides a pharmaceutical composition comprising a radiolabeled compound disclosed herein and a pharmaceutically acceptable excipient.

In a further aspect, the present invention provides a method of diagnostic imaging of monoacylglycerol lipase (MAGL) in the brain of a mammal, comprising:
(a) administering to the mammal a detectable quantity of a radiolabeled compound disclosed herein or of a pharmaceutical composition disclosed herein; and
(b) detecting the radiolabeled compound when associated with MAGL.

In a further aspect, the present invention provides the use of a radiolabeled compound disclosed herein for diagnostic imaging of monoacylglycerol lipase (MAGL) in the brain of a mammal.

In a further aspect, the present invention provides the use of a radiolabeled compound disclosed herein for the preparation of a medicament for the diagnostic imaging of monoacylglycerol lipase (MAGL) in the brain of a mammal.

### EXAMPLES

The invention will be more fully understood by reference to the following examples. The claims should not, however, be construed as limited to the scope of the examples.

The following abbreviations are used in the present text:

AcOH=acetic acid, ACN=acetonitrile, BEMP=2-tert-butylimino-2-diethylamino-1,3-dimethylperhydro-1,3,2-diazaphosphorine, Boc=tert-butyloxycarbonyl, CAS RN=chemical abstracts registration number, Cbz=benzyloxycarbonyl, Cs₂CO₃₌cesium carbonate, CO=carbon monoxide, CuCl=copper(I) chloride, CuCN=copper(I) cyanide, CuI=copper(I) iodide, DMAP=4-dimethylaminopyridine, DME=dimethoxyethane, DMEDA=N,N′-dimethylethylenediamine, DMF=N,N-dimethylformamide, DIPEA=N,N-diisopropylethylamine, dppf=1,1 bis(diphenyl phosphino)ferrocene, EDC.HCl=N-(3-dimethylaminopropyl)-N′-ethylcarbodiimide hydrochloride, EI=electron impact, ESI=electrospray ionization, EtOAc=ethyl acetate, EtOH=ethanol, h=hour(s), FA=formic acid, H₂O=water, H₂SO₄=sulfuric acid, Hal=halogen, HATU=1-[bis(dimethylamino)methylene]-1H-1,2,3-triazolo[4,5-b]pyridinium-3-oxide hexafluorophosphate, HBTU=O-benzotriazole-N,N,N′,N′-tetramethyl-uronium-hexafluoro-phosphate, HCl=hydrogen chloride, HOBt=1-hydroxy-1H-benzotriazole; HPLC=high performance liquid chromatography, iPrMgCl=isopropylmagnesium chloride, I₂=iodine, IPA=2-propanol, (Ir[dF(CF₃)ppy]₂(dtbpy))PF₆=[4,4′-bis(1,1-dimethylethyl)-2,2′-bipyridine-N1,N1′]bis[3,5-difluoro-2-[5-(trifluoromethyl)-2-pyridinyl-N]phenyl-C]Iridium(III) hexafluorophosphate, ISP=ion spray positive (mode), ISN=ion spray negative (mode), K₂CO₃=potassium carbonate, KHCO₃=potassium bicarbonate, KI=potassium iodide, KOH=potassium hydroxide, K₃PO₄=potassium phosphate tribasic, LiAlH₄ or LAH=lithium aluminium hydride, LiHMDS=lithium bis(trimethylsilyl)amide, LiOH=lithium hydroxide, MgSO₄=magnesium sulfate, min=minute(s), mL=milliliter, MPLC=medium pressure liquid chromatography, MS=mass spectrum, NaH=sodium hydride, NaHCO₃=sodium hydrogen carbonate, NaNO₂=sodium nitrite, NaOH=sodium hydroxide, Na₂CO₃=sodium carbonate, Na₂SO₄=sodium sulfate, Na₂S₂O₃=sodium thiosulfate, NBS=N-bromosuccinimide, nBuLi=n-butyllithium, NEt₃=triethylamine (TEA), NH₄Cl=ammonium chloride, NiCl₂ glyme=Nickel(II) chloride ethylene glycol dimethyl ether complex, NMP=N-methyl-2-pyrrolidone, OAc=Acetoxy, T₃P=propylphosphonic anhydride, P2O5=phosphorus pentoxide, PE=petroleum ether, PG=protective group, Pd—C=palladium on activated carbon, PdCl₂(dppf)-CH₂Cl₂=1,1′-bis(diphenylphosphino)ferrocene-palladium(II)dichloride dichloromethane complex, Pd₂(dba)₃=tris(dibenzylideneacetone)dipalladium(0), Pd(OAc)₂=palladium(II) acetate, Pd(OH)₂=palladium hydroxide, Pd(PPh₃)₄=tetrakis(triphenylphosphine)palladium(0), PTSA=p-toluenesulfonic acid, R=any group, RT=room temperature, SFC=Supercritical Fluid Chromatography, S-PHOS=2-dicyclohexylphosphino-2′,6′-dimethoxybiphenyl, T3P=propylphosphonic anhydride, TBAI=tetra butyl ammonium iodine, TBME=tert-butyl methyl ether, TEA=triethylamine, TFA=trifluroacetic acid, THF=tetrahydrofuran, TMEDA=N,N,N′,N′-tetramethylethylenediamine, ZnCl₂=zinc chloride, Xantphos=4,5-Bis(diphenylphosphino)-9,9-dimethylxanthene.

### General Methods for ³H

Reactions with tritium gas were performed on a stainless steel manifold purchased from RC Tritec AG (Teufen, Switzerland). [³H]Methyl nosylate was purchased from RC Tritec AG (Teufen, Switzerland) as a solution in toluene. Liquid scintillation counting was accomplished using a HIDEX 300 SL and ULTIMATE GOLD cocktail (PerkinElmer Inc., Waltham, Mass., USA). Analytical HPLC was performed using an Agilent 1200 series HPLC system using an XBridge Phenyl, C8 or C18 column (4.6 mm×150 mm, 3.5 μm), injected activity: 1 μCi. Radiochemical purity was measured using the β Radioactivity HPLC detector RAMONA with internal solid scintillator (Raytest, Straubenhardt, Germany). Preparative HPLC was performed on a Gilson PLC 2050 instrument (Middleton, Mich., USA). Molar activity was determined by mass spectrometric isotopic peak intensity distribution, using 4000QTRAP system (AB Sciex GmbH, Zug, CH), flow injection mode with a CTC PAL, and an Agilent 1100 microLC pump without any separation.

### General Procedure for ¹¹C Urea Synthesis

[¹¹C]CO₂ produced by ¹⁴N (p, α) ¹¹C nuclear reaction was trapped from the gas target in a stainless steel loop using liquid nitrogen. Upon warming, the [¹¹C]CO₂ in a stream of argon gas was passed through a drying column of P₂O₅, and then bubbled into a reaction vial with (4aR,8aS)-4a,5,6,7,8,8a-hexahydro-4H-pyrido[4,3-b][1,4]oxazin-3-one (BB1) (0.672 mg, 4.3 μmol) and 2-tert-butylimino-2-diethylamino-1,3-dimethylperhydro-1,3,2-diazaphosphorine (BEMP, CAS NR 98015-45-3) (5 μL, 17.3 μmol) in 100 μL anhydrous DMF. After 2 min, 0.2% POCl₃ (v/v) in anhydrous ACN (100 μL, 2.15 μmol) was added into reaction mixture. One minute later, the corresponding azetidine (4.7 μmol) in 100 μL degassed ACN was added into the reaction mixture, and further stirred at room temperature for another 2 min. The reaction was diluted by 2 mL H₂O, and purified by semi-preparative HPLC (Column: ACE 5 C18-300 250×10.0 mm; Mobile phase: MeCN/0.1% H₃PO₄). The desired product was collected, diluted with 8 mL H₂O and loaded on a pre-activated C-18 light cartridge. After washing the cartridge with 5 mL H₂O, the radioactivity was eluted by 0.5 mL ethanol, and formulated in 0.15 M PBS (9.5 mL) in a sterile vial. Injection with and without the reference were carried out in analytical HPLC (Column: Agilent Zorbax XDB-C18 3.5 μm, 4.6×75 mm; Mobile phase: ACN/0.1% H₃PO₄) to confirm the identity and the radiochemical purity of the final product. Molar activity was calculated based on a standard curve established previously.

### Synthesis of Building Block BB1

### (4aR,8aS)-4a,5,6,7,8,8a-Hexahydro-4H-pyrido[4,3-b][1,4]oxazin-3-one

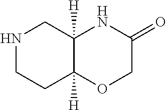 [SMILES: O=C1CO[C@H]2CCNC[C@H]2N1]

The enantiomers of rac-(4aR,8aS)-hexahydro-2H-pyrido[4,3-b][1,4]oxazin-3(4H)-one dihydrochloride (500 mg, 2.18 mmol, ChemBridge Corporation) were separated by preparative chiral HPLC (ReprosilChiral NR column) using an isocratic mixture of EtOH (containing 0.05% of NH4OAc): n-heptane (30:70).

First eluting enantiomer: (+)-cis-4a,5,6,7,8,8a-Hexahydro-4H-pyrido[4,3-b][1,4]oxazin-3-one (BB1). Yellow solid (0.150 g; 44.0%). MS (ESI): m/z=157.1 [M+H]⁺.

Second eluting enantiomer: (−)-cis-4a,5,6,7,8,8a-Hexahydro-4H-pyrido[4,3-b][1,4]oxazin-3-one. Yellow solid (0.152 g; 44.6%). MS (ESI): m/z=157.1 [M+H]⁺.

### Synthesis of Building Block BB2

### 4-Nitrophenyl (4aR,8aS)-3-oxohexahydro-2H-pyrido[4,3-b][1,4]oxazine-6(5H)-carboxylate

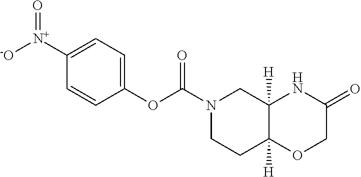

To a suspension of (4aR,8aS)-4a,5,6,7,8,8a-hexahydro-4H-pyrido[4,3-b][1,4]oxazin-3-one (3.0 g, 19 mmol) in DCM (36 mL) was added DIPEA (2.48 g, 3.35 mL, 19.2 mmol) and the mixture was cooled down in an ice-bath. To the suspension was added 4-nitrophenyl carbonochloridate (4.26 g, 21.1 mmol) in portions over 10 min and the mixture was stirred at ice-bath temperature for 15 min followed by stirring at RT for 3.25 h. The yellow solution was poured on aq. sat. Na₂CO₃ solution (30 mL) and DCM (20 mL) and the layers were separated. The aqueous layer was extracted twice with DCM (20 mL). The organic layers were washed with water (2×20 mL), dried over MgSO₄, filtered and evaporated to get the crude product as a yellow foam. The product was purified by re-crystallization from an EtOAc/DCM mixture. Further product was isolated from the mother liquor by purification with SFC (Column IB//20% MeOH). Combined yield 4.78 g (77%), light yellow solid. MS (ESI): m/z=322.2 [M+H]⁺.

### Example 1

### (4aR,8aS)-6-[3-[2-[2-Fluoro-6-(trifluoromethyl)phenyl]ethyl]azetidine-1-carbonyl]-4,4a,5,7,8,8a-hexahydropyrido[4,3-b][1,4]oxazin-3-one

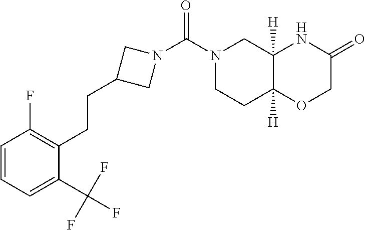 [SMILES: O=C1CO[C@H]2CCN(C(=O)N3CC(CCc4c(F)cccc4C(F)(F)F)C3)C[C@H]2N1]

To a solution of 4-nitrophenyl (4aR,8aS)-3-oxohexahydro-2H-pyrido[4,3-b][1,4]oxazine-6(5H)-carboxylate (BB2) (42 mg, 131 μmol) and DIPEA (42.3 mg, 57.1 μL, 327 μmol) in ACN (0.5 mL) was added 3-(2-fluoro-4-(trifluoromethyl)phenethyl)azetidine 4-methylbenzenesulfonate (60.3 mg, 144 μmol) and the yellow solution was stirred at RT overnight. To the reaction solution was added silica gel and the mixture was evaporated. The compound was purified by silica gel chromatography on a 4 g column using an MPLC system eluting with a gradient of n-heptane:EtOAc/ethanol 3/1 (70:30 to 10:90). A second purification on a preparative HPLC (Gemini NX column) using a gradient of ACN:H₂O (containing 0.1% formic acid) (20:80 to 98:2) afforded the title compound as colorless foam (0.038 g, 68%). MS (ESI): m/z=430.2 [M+H]⁺.

Intermediates:

### a) Diethyl (2-fluoro-6-(trifluoromethyl)benzyl)phosphonate

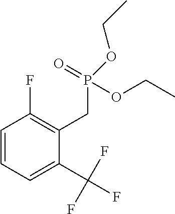 [SMILES: CCOP(=O)(Cc1c(F)cccc1C(F)(F)F)OCC]

A solution of 2-(bromomethyl)-1-fluoro-3-(trifluoromethyl)benzene (1.50 g, 5.84 mmol) in triethyl phosphite (2.42 g, 2.5 ml, 14.6 mmol) was stirred at reflux for 3 h. The clear and colorless mixture was straight applied to a silica gel column. The compound was purified by silica gel chromatography on a 40 g column (n-heptane:EtOAc 0-100%). 1.99 g (quant.), colorless oil. MS (ESI): m/z=315.1 [M+H]⁺.

### b) tert-Butyl 3-[(E)-2-[2-fluoro-6-(trifluoromethyl)phenyl]ethenyl]azetidine-1-carboxylate

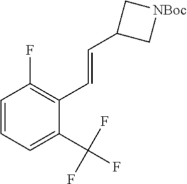 [SMILES: CC(C)(C)OC(=O)N1CC(/C=C/c2c(F)cccc2C(F)(F)F)C1]

To a solution of diethyl (2-fluoro-6-(trifluoromethyl)benzyl)phosphonate (1.50 g, 4.77 mmol) in THF (20 ml) at 0° C. was added NaH 55% in mineral oil (208 mg, 4.77 mmol). The mixture was stirred at 0° C. for 30 min. To the mixture was added tert-butyl 3-formylazetidine-1-carboxylate (CAS NR 398489-26-4) (884 mg, 4.77 mmol) and stirring was continued at RT for 4.5 h. The reaction mixture was extracted EtOAc (2×)/H₂O and the layers were separated. The combined organic layers were dried over MgSO₄, filtered, absorbed on silica gel and evaporated. The compound was purified by silica gel chromatography (n-hept:EtOAc 0-100%). 900 mg (55%), colorless oil. MS (ESI): m/z=290.2 [M-C₄H₉+H]⁺.

### c) tert-Butyl 3-[2-[2-fluoro-6-(trifluoromethyl)phenyl]ethyl]azetidine-1-carboxylate

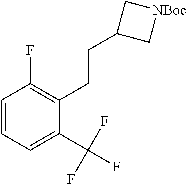 [SMILES: CC(C)(C)OC(=O)N1CC(CCc2c(F)cccc2C(F)(F)F)C1]

tert-Butyl (E)-3-(2-fluoro-6-(trifluoromethyl)styryl)azetidine-1-carboxylate (2.80 g, 8.11 mmol) was combined with MeOH (50 ml). Pd/C 10% (280 mg) was added and the reaction mixture was stirred under hydrogen overnight. The catalyst was filtered off, the solvent was evaporated and the product dried and used as such in the following step.

### d) 3-(2-Fluoro-6-(trifluoromethyl)phenethyl)azetidine 4-methylbenzenesulfonate

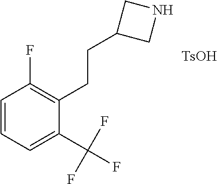

To an solution of tert-butyl 3-(2-fluoro-6-(trifluoromethyl)phenethyl)azetidine-1-carboxylate (800 mg, 2.3 mmol) in EtOAc (5 ml) was added 4-methylbenzenesulfonic acid monohydrate (438 mg, 2.3 mmol) and the mixture was heated at reflux for 1 h. The clear, colorless solution was allowed to cool down to RT. The solvent was evaporated. Upon cooling over night at 4° a solid was formed. The crystals were washed with Et₂O. Half of this material was dissolved in AcOEt/DCM and treated with pentane/Et₂O. The white crystals were isolated and dried at HV. 400 mg (41%). MS (ESI): m/z=248.1 [M+H]⁺.

### Example [³H]1

### [³H](4aR,8aS)-6-[3-[2-[2-Fluoro-6-(trifluoromethyl)phenyl]ethyl]azetidine-1-carbonyl]-4,4a,5,7,8,8a-hexahydropyrido[4,3-b][1,4]oxazin-3-one

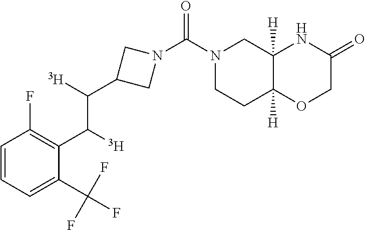 [SMILES: [3H]C(c1c(F)cccc1C(F)(F)F)C([3H])C1CN(C(=O)N2CC[C@@H]3OCC(=O)N[C@@H]3C2)C1]

In a 4 mL tritiation flask (4aR,8aS)-6-(3-((E)-2-fluoro-6-(trifluoromethyl)styryl)azetidine-1-carbonyl)hexahydro-2H-pyrido[4,3-b][1,4]oxazin-3(4H)-one (2.0 mg, 4.68 μmol) and palladium on activated charcoal (10%) (4.98 mg, 4.68 μmol) were mixed in ethanol (1 mL). The flask was attached to a RC Tritec tritium manifold and degassed by three freeze-thaw cycles. Tritium gas was introduced, and the suspension was vigorously stirred for 4 h under an atmosphere of tritium gas at 610 mbar. The solution was cooled by liquid nitrogen and the excess tritium gas in the reaction vessel was reabsorbed on an uranium trap for waste-tritium. The solvent was lyophilized off, and labile tritium was removed by lyophilization with MeOH (3×0.5 mL). The remaining black residue was suspended in ethanol (10 ml) and filtered over a 17 mm Titan HPLC filter (0.45 μm, PTFE) to provide 9.32 GBq (252 mCi) of a crude product in a radiochemical purity of 94%. The crude product was purified by preparative HPLC (XBridge Phenyl column, 5 μm, 10 mm×250 mm) using [A] ACN and [B] H₂O as eluents (gradient 1-12 min from [A]:[B] 3:7 to 7:3, 12-12.5 min to 9:1, 15-18 min 9:1 to 3:7 run time 20 min, detection at 215 nm, oven temperature at 55° C.) at a flow rate of 10 mL/min. The combined pure HPLC fractions were concentrated and the product dissolved and stored in EtOH (50 ml). An amount of 7.8 GBq (210 mCi) of the title compound was obtained with a radiochemical purity of 99.5% and a molar activity of 1.7 TBq/mmol (44.9 Ci/mmol), determined by MS spectrometry. The identity of the labeled compound was confirmed by MS and by co-injection of the cold reference standard with the radiolabeled material. MS: m/z=430.2 [M(H)+H]⁺ (4%), 432.2 [M(³H)+H]⁺ (35%), 434.2 [M(³H₂)+H]⁺ (60%).

Intermediates:

### a) 3-[(E)-2-[2-Fluoro-6-(trifluoromethyl)phenyl]vinyl]azetidine

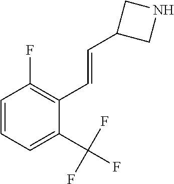 [SMILES: Fc1cccc(C(F)(F)F)c1/C=C/C1CNC1]

To a solution of tert-butyl 3-[(E)-2-[2-fluoro-6-(trifluoromethyl)phenyl]ethenyl]azetidine-1-carboxylate (900 mg, 2.61 mmol) in DCM (373 μl) was added TFA (2.38 g, 1.61 ml, 20.8 mmol) and the reaction mixture was stirred at r.t for 3 h. The reaction mixture was extracted EtOAc (2×)/KHCO₃. The combined organic phases were dried over sodium sulfate and evaporated down to dryness to afford the title compound (795 mg, quant., 84% purity) as off-white solid. MS (ESI): m/z=474.1 [M+H]⁺.

### d) (4aR,8aS)-6-(3-((E)-2-Fluoro-6-(trifluoromethyl)styryl)azetidine-1-carbonyl)hexahydro-2H-pyrido[4,3-b][1,4]oxazin-3(4H)-one

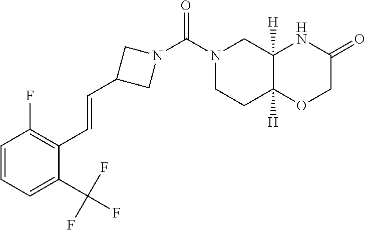 [SMILES: O=C1CO[C@H]2CCN(C(=O)N3CC(/C=C/c4c(F)cccc4C(F)(F)F)C3)C[C@H]2N1]

A solution of (4aR,8aS)-4a,5,6,7,8,8a-hexahydro-4H-pyrido[4,3-b][1,4]oxazin-3-one (BB1) (220 mg, 816 μmol) and sodium bicarbonate (274 mg, 3.26 mmol) in DCM (13 ml) was cold to 0° C. Then triphosgene (169 mg, 571 μmol) was added and the mixture was stirred at RT overnight. The mixture was cooled in an ice-bath and (E)-3-(2-fluoro-6-(trifluoromethyl)styryl)azetidine (200 mg, 816 μmol) and DIPEA (422 mg, 570 μl, 3.26 mmol) were added. The suspension was stirred at RT for 2 h. The reaction mixture was extracted with EtOAc/H₂O, dried over Na₂SO₄, and the solvent was removed under reduced pressure. The product was purified by preparative HPLC, yielding the title compound (354 mg, 98%, 97% purity) as white foam. MS (ESI): m/z=428.2 [M+H]⁺.

### Example [¹¹C]1

### [¹¹C](4aR,8aS)-6-[3-[2-[2-Fluoro-6-(trifluoromethyl)phenyl]ethyl]azetidine-1-carbonyl]-4,4a,5,7,8,8a-hexahydropyrido[4,3-b][1,4]oxazin-3-one

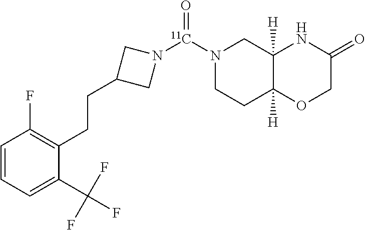 [SMILES: O=C1CO[C@H]2CCN([11C](=O)N3CC(CCc4c(F)cccc4C(F)(F)F)C3)C[C@H]2N1]

The general procedure described above was applied with 3-(2-fluoro-6-(trifluoromethyl)phenethyl)azetidine (formic acid salt, 4.7 μmol, 1.16 mg) as a precursor at the third step. The product was obtained with a radiochemical purity above 99% and a molar activity of 66-126 GBq/μmol.

### Example 2

### (4aR,8aS)-6-[6-[(2-Fluoro-6-methoxy-phenyl)methyl]-2-azaspiro[3.3]heptane-2-carbonyl]-4,4a,5,7,8,8a-hexahydropyrido[4,3-b][1,4]oxazin-3-one

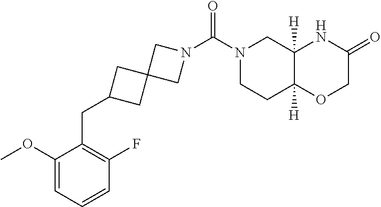 [SMILES: COc1cccc(F)c1CC1CC2(C1)CN(C(=O)N1CC[C@@H]3OCC(=O)N[C@@H]3C1)C2]

A round-bottom flask was heat gun-dried under HV, back filled with argon and charged with bis(trichloromethyl) carbonate (39.9 mg, 134 μmol) and sodium bicarbonate (64.5 mg, 768 μmol). DCM (10 ml) was added to give a suspension. (4aR,8aS)-4a,5,6,7,8,8a-Hexahydro-4H-pyrido[4,3-b][1,4]oxazin-3-one (0.03 g, 192 μmol) was added to the suspension at 0° C. The mixture was stirred at 0° C. for 5 min and at RT for 20 hours. 6-(2-fluoro-6-methoxybenzyl)-2-azaspiro[3.3]heptane 2,2,2-trifluoroacetate (67.1 mg, 192 μmol) and DIPEA (99.3 mg, 134 μl, 768 μmol) were added. The resulting off-white suspension was stirred at RT for 1 h. The reaction mixture was poured into H₂O (20 mL) and extracted with DCM (2×50 mL). The organic layers were combined, washed with brine, dried over Na₂SO₄ and concentrated under vacuum. The crude material was purified by flash chromatography (silica gel, 20 g, 0% to 10% MeOH in DCM). MS (ESI): m/z=418.2 [M+H]⁺.

Intermediates:

### a) (2-Fluoro-6-methoxybenzyl)triphenylphosphonium bromide

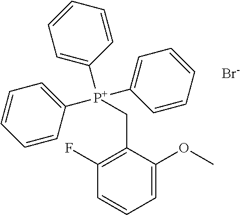 [SMILES: COc1cccc(F)c1C[P+](c1ccccc1)(c1ccccc1)c1ccccc1 ; [Br-]]

Under Ar, PPh₃ (1.20 g, 4.57 mmol) was dissolved in ACN (10 mL) and 2-(bromomethyl)-1-fluoro-3-methoxybenzene (1.00 g, 4.57 mmol) was added. The mixture was stirred at 80° C. for 3 h. The resulting suspension was allowed to cool to RT. TBME (100 mL) was added and the mixture was stirred at RT for 30 min. The solid was filtrated and washed with TBME, then it was dried under high vacuum to yield the title compound (2.19 g, 100%) as white solid. MS (ESI): m/z=401.2 M⁺.

### b) tert-Butyl 6-(2-fluoro-6-methoxybenzylidene)-2-azaspiro[3.3]heptane-2-carboxylate

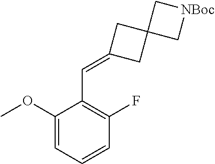 [SMILES: COc1cccc(F)c1C=C1CC2(C1)CN(C(=O)OC(C)(C)C)C2]

Under Ar at −78° C., (2-fluoro-6-methoxybenzyl)triphenylphosphonium bromide (0.5 g, 1.04 mmol) was dissolved in dry THF (5 ml) and LHMDS (2.08 ml, 2.08 mmol) was added. The reaction mixture was stirred at −78° C. for 2 h. Then at RT, tert-butyl 6-oxo-2-azaspiro[3.3]heptane-2-carboxylate (CAS NR 1181816-12-5) (219 mg, 1.04 mmol) was added and the mixture was stirred at 85° C. overnight. TBDME was added. The resulting precipitate was filtrated off (TPPO). The filtrate was concentrated and purified by flash chromatography (silica gel, 20 g, 0% to 80% EtOAc in heptane). 78 mg (22%), colorless oil. MS (ESI): m/z=278.2 [M-C₄H₈+H]⁺.

### c) tert-Butyl 6-(2-fluoro-6-methoxybenzyl)-2-azaspiro[3.3]heptane-2-carboxylate

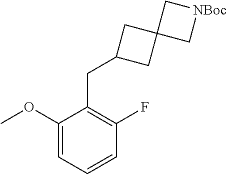 [SMILES: COc1cccc(F)c1CC1CC2(C1)CN(C(=O)OC(C)(C)C)C2]

tert-Butyl 6-(2-fluoro-6-methoxybenzylidene)-2-azaspiro[3.3]heptane-2-carboxylate (78.0 mg, 234 μmol) was combined with EtOAc (2 ml) to give a light yellow solution. The flask was purged and backfilled with Ar (3×). Pd—C 10% (24.9 mg, 23.4 μmol) was added and the reaction was stirred under H₂ for 2 h. The reaction mixture was filtered through a Celite pad, wash with EtOAc and dried on the high vacuum. MS (ESI): m/z=280.2 [M-C₄H₈+H]⁺.

### d) 6-(2-Fluoro-6-methozybenzyl)-2-azaspiro[3.3]heptane 2,2,2-trifluoroacetate

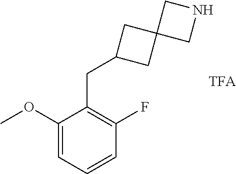 [SMILES: COc1cccc(F)c1CC1CC2(CNC2)C1]

To a solution of tert-butyl 6-(2-fluoro-6-methoxybenzyl)-2-azaspiro[3.3]heptane-2-carboxylate (76.2 mg, 227 μmol) in DCM (2 ml) was added 2,2,2-trifluoroacetic acid (130 mg, 86.9 μl, 1.14 mmol). The resultant reaction mixture was stirred at RT for 2 h. The reaction mixture was concentrated under vacuum and then by high vacuum, adding Tol for azeotropical removal of volatiles. 81 mg (quant.), colorless oil. MS (ESI): m/z=236.2 [M+H]⁺.

### Example [³H]2

### [³H](4aR,8aS)-6-[6-[(2-Fluoro-6-methoxy-phenyl)methyl]-2-azaspiro[3.3]heptane-2-carbonyl]-4,4a,5,7,8,8a-hexahydropyrido[4,3-b][1,4]oxazin-3-one

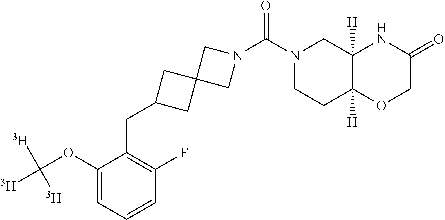 [SMILES: [3H]C([3H])([3H])Oc1cccc(F)c1CC1CC2(C1)CN(C(=O)N1CC[C@@H]3OCC(=O)N[C@@H]3C1)C2]

To [³H]methyl nosylate (1.85 GBq, 50 mCi, 0.61 μmol) was added a solution of phenol precursor (4aR,8aS)-6-[6-[(2-fluoro-6-hydroxy-phenyl)methyl]-2-azaspiro[3.3]heptane-2-carbonyl]-4,4a,5,7,8,8a-hexahydropyrido[4,3-b][1,4]oxazin-3-one (0.54 mg, 1.34 μmol) in THF (150 μl). Cesium carbonate (1.1 mg, 3.36 μmol) was added, and the reaction mixture was stirred for 2 h at room temperature. Then H₂O was added and the solvent was removed under a stream of argon. The crude product was purified by HPLC (Sunfire C18 OBD, 10×250 mm, ACN [A]/H₂O [B], gradient: 1-12 min 3:7 to 9:1 [A]:[B], 15-16 min 9:1 to 3:7, run time 20 min, flow rate 8 ml/min, 230 nm, oven temperature 60° C.). The pure fractions were combined and the solvent was lyophilized off. The pure tritium-labeled compound was dissolved and stored as ethanolic solution (10 ml) and was obtained in an amount of 296 MBq (8 mCi). The radiochemical purity of >99.5% was determined by radio-HPLC and the specific activity of 3.0 TBq/mmol (81 Ci/mmol) by mass spectrometry (MS). The identity of the labeled compound was confirmed by HPLC (by co-injecting the unlabeled reference standard) and by MS. MS: m/z=418.2 [M(H)+H]⁺ (4%), 420.2 [M(³H)+H]⁺ (0%), 422.2 [M(³H₂)+H]⁺ (4%), 424.2 [M(³H₃)+H]⁺ (92%).

Intermediates:

### a) 2-((2-Azaspiro[3.3]heptan-6-yl)methyl)-3-fluorophenol

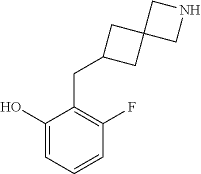 [SMILES: Oc1cccc(F)c1CC1CC2(CNC2)C1]

tert-Butyl 6-(2-fluoro-6-methoxybenzyl)-2-azaspiro[3.3]heptane-2-carboxylate (350 mg, 1.04 mmol) was combined with DCM (7 ml) to give a colorless solution. BBr₃ (523 mg, 197 μl, 2.09 mmol) was added at 0° C. The reaction was stirred at RT overnight. The reaction mixture was quenched by addition of sat. aq. NaHCO₃ and extracted with EtOAc/THF. The organic layers were combined, washed with brine, dried over Na₂SO₄ and concentrated under vacuum. 231 mg (100%), yellow solid. MS (ESI): m/z=222.2 [M+H]⁺.

### a) (4aR,8aS)-6-[6-[(2-Fluoro-6-hydroxy-phenyl)methyl]-2-azaspiro[3.3]heptane-2-carbonyl]-4,4a,5,7,8,8a-hexahydropyrido[4,3-b][1,4]oxazin-3-one

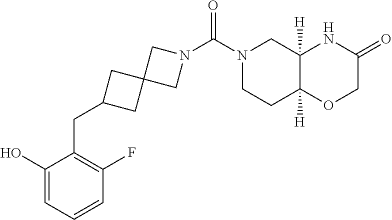 [SMILES: O=C1CO[C@H]2CCN(C(=O)N3CC4(CC(Cc5c(O)cccc5F)C4)C3)C[C@H]2N1]

(4aR,8aS)-4a,5,6,7,8,8a-Hexahydro-4H-pyrido[4,3-b][1,4]oxazin-6-ium-3-one; (2S,3S)-2,3-bis[(4-methylbenzoyl)oxy]butanedioic acid; (2S,3S)-4-hydroxy-2,3-bis[(4-methylbenzoyl)oxy]-4-oxobutanoate; hydrate (salt of BB1) (300 mg, 431 μmol) was suspended in ACN (7 ml) and triethylamine (305 mg, 420 μl, 3.01 mmol) was added under stirring at RT. Then bis(1,2,4-triazol-1-yl)methanone (70.7 mg, 431 μmol) was added in one portion. The reaction mixture was stirred at RT for 2 h. 2-((2-Azaspiro[3.3]heptan-6-yl)methyl)-3-fluorophenol (114 mg, 517 μmol) dissolved in ACN (350 μl) was added dropwise at RT. The reaction mixture was heated at 50° C. over 10 min and stirred at that temperature for 3 h. The reaction mixture was cooled down to r.t, quenched with 2 mL H₂O, and then extracted with 4 mL TBME. The organic layer was washed with 2 mL 5% NaHCO₃ and then with 1 mL 0.5M HCl. The organic layer was washed with brine, dried over Na₂SO₄ and concentrated under vacuum (yellow oil, 8.15 g). The crude material was purified by flash chromatography (silica gel, 20 g, 0% to 10% MeOH in DCM), concentrated, dissolved in ACN/H₂O and lyophilized. 134 mg (94%), white solid. MS (ESI): m/z=404.3 [M+H]⁺.

### Example 3

### (4aR,8aS)-6-[3-[2-(2-Fluoro-4-methyl-phenyl)ethyl]azetidine-1-carbonyl]-4,4a,5,7,8,8a-hexahydropyrido[4,3-b][1,4]oxazin-3-one

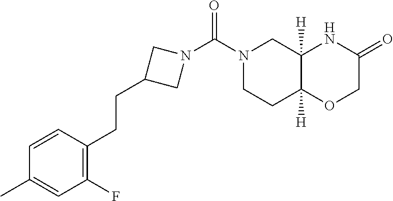 [SMILES: Cc1ccc(CCC2CN(C(=O)N3CC[C@@H]4OCC(=O)N[C@@H]4C3)C2)c(F)c1]

A mixture of 3-[2-(2-fluoro-4-methyl-phenyl)ethyl]azetidine; 2,2,2-trifluoroacetic acid (100 mg, 0.330 mmol), (4-nitrophenyl) (4aR,8aS)-3-oxo-4,4a,5,7,8,8a-hexahydropyrido[4,3-b][1,4]oxazine-6-carboxylate (BB2) (125 mg, 0.390 mmol) and DIPEA (126 mg, 0.980 mmol) in ACN (6 mL) was stirred at 80° C. for 12 h. The mixture was concentrated by reduced pressure, the residue was purified by Prep-HPLC (NH₄HCO₃), then lyophilized to give (4aR,8aS)-6-[3-[2-(2-fluoro-4-methyl-phenyl)ethyl]azetidine-1-carbonyl]-4,4a,5,7,8,8a-hexahydropyrido[4,3-b][1,4]oxazin-3-one (50 mg, 41%) as white solid. MS (ESI): m/z=376.0 [M+H]⁺.

Intermediates:

### a) tert-Butyl 3-[2-(2-fluoro-4-methyl-phenyl)ethynyl]azetidine-1-carboxylate

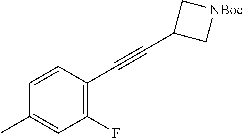 [SMILES: Cc1ccc(C#CC2CN(C(=O)OC(C)(C)C)C2)c(F)c1]

To a solution of tert-butyl 3-ethynylazetidine-1-carboxylate (CAS NR 287193-01-5) (1.00 g, 5.52 mmol) and 4-bromo-3-fluorotoluene (CAS NR 452-74-4) (1.25 g, 6.62 mmol) in dry THF (20 mL) at 25° C., was added [Pd(PPh₃)₄] (531 mg, 0.460 mmol), CuI (88 mg, 0.46 mmol) and TEA (4.64 g, 46.0 mmol). The mixture was purged by N₂ for 1 min, then it was stirred at 60° C. under N₂ atmosphere for 12 h. The mixture was poured into NH₄Cl (sat., 50 mL), extracted with EtOAc (3×30 mL) and the combined organic layers were dried with anhydrous Na₂SO₄. After solvent evaporation the residue was purified by flash chromatography on silica gel (PE:EtOAc=20:1 to 10:1) to obtain the tert-butyl 3-[2-(2-fluoro-4-methyl-phenyl)ethynyl]azetidine-1-carboxylate (650 mg, 41%) as colorless oil. ¹H NMR (400 MHz, CHLOROFORM-d) δ=7.33-7.28 (m, 1H), 6.94-6.85 (m, 2H), 4.26-4.19 (m, 2H), 4.05 (dd, J=6.4, 8.1 Hz, 2H), 3.66-3.49 (m, 1H), 2.36 (s, 3H), 1.46 (s, 9H).

### b) tert-Butyl 3-[2-(2-fluoro-4-methyl-phenyl)ethyl]azetidine-1-carboxylate

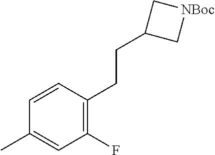 [SMILES: Cc1ccc(CCC2CN(C(=O)OC(C)(C)C)C2)c(F)c1]

To a solution of tert-butyl 3-[2-(2-fluoro-4-methyl-phenyl)ethynyl]azetidine-1-carboxylate (500 mg, 1.73 mmol) in EtOAc (10 mL) at 25° C., was added Pd/C 10% (250 mg, 1.73 mmol). The mixture was stirred at 40° C. under a balloon of H₂ (15 psi) for 6 h. The reaction mixture was combined with a previous batch (0.2 mmol scale), the mixture was filtered through a pad of celite, the filtrate was concentrated under reduced pressure and the residue was dried under vacuum. 350 mg, colorless oil. MS (ESI): m/z=238.1 [M-C₄H₈H]⁺.

### c) 3-[2-(2-Fluoro-4-methyl-phenyl)ethyl]azetidine; 2,2,2-trifluoroacetic acid

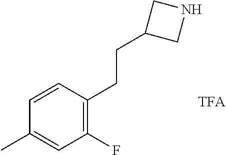

To a solution of tert-butyl 3-[2-(2-fluoro-4-methyl-phenyl)ethyl]azetidine-1-carboxylate (350 mg, 1.19 mmol) in dry DCM (10 mL) at 25° C., was added TFA (1.0 mL, 1.19 mmol). The mixture was stirred at 25° C. for 12 h. The reaction mixture was concentrated by reduced pressure and the residue was dried under vacuum to obtain 3-[2-(2-fluoro-4-methyl-phenyl)ethyl]azetidine; 2,2,2-trifluoroacetic acid (260 mg, 71%) as colorless oil. MS (ESI): m/z=194.0 [M+H]⁺.

### Example [³H]3

### [³H](4aR,8aS)-6-[3-[2-(2-Fluoro-4-methyl-phenyl)ethyl]azetidine-1-carbonyl]-4,4a,5,7,8,8a-hexahydropyrido[4,3-b][1,4]oxazin-3-one

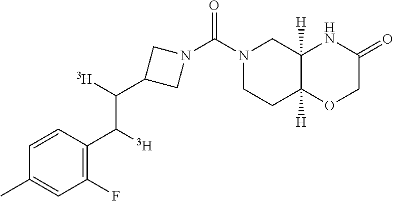 [SMILES: [3H]C(c1ccc(C)cc1F)C([3H])C1CN(C(=O)N2CC[C@@H]3OCC(=O)N[C@@H]3C2)C1]

In a 4 mL tritiation flask, (4aR,8aS)-6-[3-[(E)-2-(2-fluoro-4-methyl-phenyl)vinyl]azetidine-1-carbonyl]-4,4a,5,7,8,8a-hexahydropyrido[4,3-b][1,4]oxazin-3-one (2.0 mg, 5.36 μmol) and palladium on activated charcoal (10% Pd basis) (6.27 mg, 5.89 μmol) were mixed in ethanol (1 mL). The flask was attached to a RC Tritec tritium manifold and degassed by three freeze-thaw cycles. Tritium gas was introduced, and the suspension was vigorously stirred for 3 h in an atmosphere of tritium at 600 mbar. The solution was cooled by liquid nitrogen and the excess tritium gas in the reaction vessel was reabsorbed on a uranium trap for waste-tritium. The solvent was lyophilized off, and labile tritium was removed by lyophilization with MeOH (3×0.5 mL).

The remaining reaction mixture was diluted with EtOH and filtered from black palladium residue. The crude product was purified by preparative HPLC (XBridge C8 column, 5 μm, 10 mm×250 mm) using [A] H₂O and [B] ACN as eluents (gradient 6-20 min from [A]:[B] 9:1 to 3:7, run time 23 min) at a flow rate of 6 mL/min. An amount of 3.3 GBq (89 mCi) of the title compound was obtained with a radiochemical purity of 99.1% and a molar activity of 1.8 TBq/mmol (49.5 Ci/mmol), determined by MS spectrometry. The identity of the labeled compound was confirmed by MS and by co-injection of the cold reference standard with the radiolabeled material. MS: m/z=376.2 [M(H)+H]⁺ (5%), 378.2 [M(³H)+H]⁺ (24%), 380.2 [M(³H₂)+H]⁺ (65%), 382.2 [M(³H₃)+H]⁺ (6%).

Intermediates:

### a) 1-(Diethoxyphosphorylmethyl)-2-fluoro-4-methyl-benzene

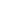

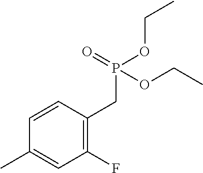 [SMILES: CCOP(=O)(Cc1ccc(C)cc1F)OCC]

A solution of 1-(bromomethyl)-2-fluoro-4-methylbenzene (1 g, 4.92 mmol) in triethyl phosphite (2.05 g, 2.14 ml, 12.3 mmol) was stirred at reflux for 2.5 h. The clear and colorless mixture was directly applied to a silica gel column. The compound was twice purified by silica gel chromatography on a 40 g column using an MPLC (ISCO) system eluting with a gradient of n-heptane:EtOAc (100:0 to 0:100) to get the desired compound as colorless liquid (1.09 g, 85%). MS (ESI): m/z=261.1 [M+H]⁺.

### b) tert-butyl (E)-3-(2-fluoro-4-methylstyryl)azetidine-1-carboxylate

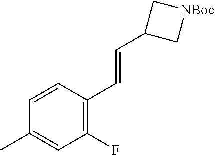 [SMILES: Cc1ccc(/C=C/C2CN(C(=O)OC(C)(C)C)C2)c(F)c1]

To an ice-cold solution of 1-(diethoxyphosphorylmethyl)-2-fluoro-4-methyl-benzene (1.09 g, 4.19 mmol) in THF (7 mL) was added NaH 55% in mineral oil (183 mg, 4.19 mmol) and the mixture was stirred at this temperature for 30 min. To the light brown mixture was added dropwise a solution of tert-butyl 3-formylazetidine-1-carboxylate (CAS NR 398489-26-4) (776 mg, 4.19 mmol) in THF (5 mL). Stirring was continued at RT overnight, then at 50° C. for 3 days. The reaction mixture was poured on sat. aq. NH₄Cl solution and EtOAc and the layers were separated. The aqueous layer was extracted with EtOAc (2×). The organic layers were dried over MgSO₄, filtered, treated with silica gel and evaporated. The compound was purified by silica gel chromatography on a 40 g column using an MPLC system eluting with a gradient of n-heptane:EtOAc (100:0 to 0:100) to get the desired compound as a colorless oil (85 mg; 7%). MS (ESI): m/z=236.2 [M-C₄H₉+H]⁺.

### c) (E)-3-(2-Fluoro-4-methylstyryl)azetidine 4-methylbenzenesulfonate

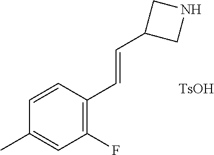

A solution of tert-butyl (E)-3-(2-fluoro-4-methylstyryl)azetidine-1-carboxylate (129 mg, 443 μmol) and 4-methylbenzenesulfonic acid hydrate (88.4 mg, 465 μmol) in EtOAc (1.5 mL) was stirred a reflux for 2 h. The suspension was cooled down at 4° C., then filtered. The filter cake was washed with EtOAc to get the desired compound as colorless solid (140 mg, 87%). MS (ESI): m/z=192.2 [M+H]⁺.

### d) (4aR,8aS)-6-[3-[(E)-2-(2-fluoro-4-methyl-phenyl)vinyl]azetidine-1-carbonyl]-4,4a,5,7,8,8a-hexahydropyrido[4,3-b][1,4]oxazin-3-one

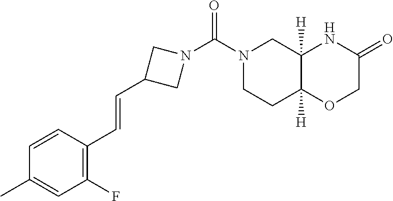 [SMILES: Cc1ccc(/C=C/C2CN(C(=O)N3CC[C@@H]4OCC(=O)N[C@@H]4C3)C2)c(F)c1]

To a suspension of (4aR,8aS)-4a,5,6,7,8,8a-hexahydro-4H-pyrido[4,3-b][1,4]oxazin-6-ium-3-one; (2S,3S)-2,3-bis[(4-methylbenzoyl)oxy]butanedioic acid; (2S,3S)-4-hydroxy-2,3-bis[(4-methylbenzoyl)oxy]-4-oxobutanoate; hydrate (salt of BB1) (259 mg, 371 μmol) in MeCN (1.5 mL) was added TEA (263 mg, 362 μL, 2.6 mmol) followed by addition of bis(1,2,4-triazol-1-yl)methanone (61 mg, 371 μmol) in one portion. The mixture was stirred at RT for 40 min. To the solution was added (E)-3-(2-fluoro-4-methylstyryl)azetidine 4-methylbenzenesulfonate (135 mg, 371 μmol) and the mixture was stirred at 50° C. for 3 h followed by stirring overnight at 70° C. After cooling down, the reaction mixture was poured on water and EtOAc and the layers were separated. The aqueous layer was extracted with EtOAc. The organic layers were washed with water, dried over MgSO₄, filtered, treated with silica gel and evaporated. The compound was purified by silica gel chromatography on a 4 g column using an MPLC system eluting with a gradient of n-heptane:EtOAc/EtOH 3/1 (100:0 to 0:100) to get the desired compound as colorless foam (108 mg, 78%). MS (ESI): m/z=374.2 [M+H]⁺.

### Example 4

### (4aR,8aS)-6-[3-[2-[4-Methoxy-2-(trifluoromethyl)phenyl]ethyl]azetidine-1-carbonyl]-4,4a,5,7,8,8a-hexahydropyrido[4,3-b][1,4]oxazin-3-one

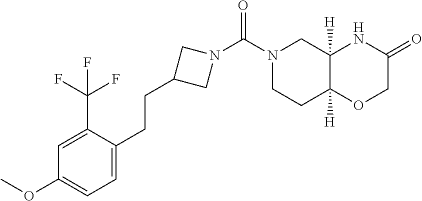 [SMILES: COc1ccc(CCC2CN(C(=O)N3CC[C@@H]4OCC(=O)N[C@@H]4C3)C2)c(C(F)(F)F)c1]

A mixture of 3-[2-[4-methoxy-2-(trifluoromethyl)phenyl]ethyl]azetidine; 2,2,2-trifluoroacetic acid (150 mg, 0.400 mmol), (4aR,8aS)-3-oxohexahydro-2H-pyrido[4,3-b][1,4]oxazine-6(5H)-carboxylate (BB2) (155 mg, 0.480 mmol) and DIPEA (156 mg, 1.21 mmol) in ACN (12 mL) was stirred at 80° C. for 12 h. The mixture was concentrated by reduced pressure, the residue was purified by Prep-HPLC (NH₄HCO₃), then lyophilized to give (4aR,8aS)-6-[3-[2-[4-methoxy-2-(trifluoromethyl)phenyl]ethyl]azetidine-1-carbonyl]-4,4a,5,7,8,8a-hexahydropyrido[4,3-b][1,4]oxazin-3-one (44 mg, 36%) as white solid. MS (ESI): m/z=442.3 [M+H]⁺.

Intermediates:

### a) tert-Butyl 3-[2-[4-methoxy-2-(trifluoromethyl)phenyl]ethynyl]azetidine-1-carboxylate

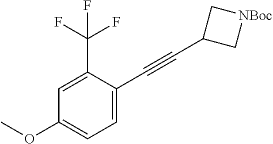 [SMILES: COc1ccc(C#CC2CN(C(=O)OC(C)(C)C)C2)c(C(F)(F)F)c1]

To a solution of tert-butyl 3-ethynylazetidine-1-carboxylate (CAS NR 287193-01-5) (800 mg, 4.41 mmol) and 3-trifluoromethyl-4-bromoanisole (CAS NR 400-72-6) (1.3 g, 5.3 mmol) in dry THF (30 mL) at 25° C. were added [Pd(PPh₃)₄] (509 mg, 0.440 mmol), CuI (84 mg, 0.44 mmol) and TEA (4.46 mg, 44.1 mmol). The mixture was purged with N₂ for 1 min, then it was stirred at 60° C. for 12 h. The mixture was poured into NH₄Cl (aq. sat., 100 mL), extracted with EtOAc (3×50 mL) and the organic layers were combined, dried with anhydrous Na₂SO₄, and concentrated under reduced pressure. The residue was purified by flash chromatography on silica gel (PE:EtOAc 20:1 to 10:1) to obtain tert-butyl 3-[2-[4-methoxy-2-(trifluoromethyl)phenyl]ethynyl]azetidine-1-carboxylate (160 mg, 8.1%) as colorless oil. MS (ESI): m/z=300.1 [M-C₄H₈+H]⁺.

### b) tert-Butyl 3-[2-[4-methoxy-2-(trifluoromethyl)phenyl]ethyl]azetidine-1-carboxylate

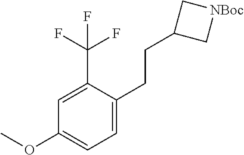 [SMILES: COc1ccc(CCC2CN(C(=O)OC(C)(C)C)C2)c(C(F)(F)F)c1]

To a solution of tert-butyl 3-[2-[4-methoxy-2-(trifluoromethyl)phenyl]ethynyl]azetidine-1-carboxylate (230 mg, 0.650 mmol) in EtOAc (10 mL) at 25° C. was added Pd/C 10% (150.0 mg) and the mixture was stirred at 40° C. under a balloon of H₂ for 12 h. The reaction mixture was filtered through a pad of Celite, the filtrate was concentrated under reduced pressure and the residue was dried under vacuum to give the title compound (180 mg, 77%) as colorless oil. MS (ESI): m/z=304.1 [M-C₄H₈+H]⁺.

### c) 3-[2-[4-Methoxy-2-(trifluoromethyl)phenyl]ethyl]azetidine; 2,2,2-trifluoroacetic acid

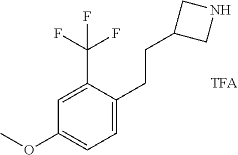 [SMILES: COc1ccc(CCC2CNC2)c(C(F)(F)F)c1]

To a solution of tert-butyl 3-[2-[4-methoxy-2-(trifluoromethyl)phenyl]ethyl]azetidine-1-carboxylate (180 mg, 0.500 mmol) in dry DCM (10 mL) at 25° C. was added TFA (1.0 mL, 1.19 mmol) and the mixture was stirred at 25° C. for 12 h. The reaction mixture was concentrated by reduced pressure and the residue was dried under vacuum to obtain 3-[2-[4-methoxy-2-(trifluoromethyl)phenyl]ethyl]azetidine; 2,2,2-trifluoroacetic acid (150 mg, 80%) as colorless oil. MS (ESI): m/z=260.1 [M+H]⁺.

### Example [³H]4

### [³H](4aR,8aS)-6-[3-[2-[4-Methoxy-2-(trifluoromethyl)phenyl]ethyl]azetidine-1-carbonyl]-4,4a,5,7,8,8a-hexahydropyrido[4,3-b][1,4]oxazin-3-one

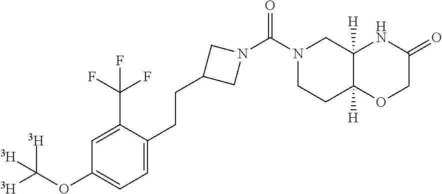 [SMILES: [3H]C([3H])([3H])Oc1ccc(CCC2CN(C(=O)N3CC[C@@H]4OCC(=O)N[C@@H]4C3)C2)c(C(F)(F)F)c1]

To a solution of [³H]methyl nosylate (1.85 GBq, 50 mCi, 0.61 μmol) in DMF (100 μl) was added the phenol precursor (4aR,8aS)-6-[3-[2-[4-hydroxy-2-(trifluoromethyl)phenyl]ethyl]azetidine-1-carbonyl]-4,4a,5,7,8,8a-hexahydropyrido[4,3-b][1,4]oxazin-3-one (0.57 mg, 1.33 μmol) dissolved in DMF (150 μl). Cesium carbonate (1 mg, 3.07 μmol) was added and the reaction mixture was stirred for 2 h at room temperature before it was treated with H₂O and TBME. After separation of the organic layer the aqueous layer was again extracted with TBME. The combined organic layers were consecutively washed with 1 N NaOH and H₂O before they were dried over sodium sulfate. After evaporation of the organic solvent the crude product was purified by HPLC (X-Terra Prep RP-18, 10×150 mm, ACN/H₂O (containing 5% of acetonitrile), 3 ml/min, 210 nm). The pure tritium-labeled compound was isolated by solid phase extraction (Sep-Pak Plus C18) and eluted from the cartridge as ethanolic solution to yield 655 MBq (17.7 mCi) of the target compound in 98.2% radiochemical purity and a specific activity of 3.03 TBq/mmol (82 Ci/mmol) as determined by mass spectrometry (MS). The identity of the labeled compound was confirmed by HPLC (by co-injecting the unlabeled reference standard) and by MS. MS: m/z=442 [M(H)+H]⁺ (4%), 444 [M(³H)+H]⁺ (0%), 446 [M(³H₂)+H]⁺ (4%), 448 [M(³H₃)+H]⁺ (92%).

Intermediate:

### a) (4aR,8aS)-6-[3-[2-[4-hydroxy-2-(trifluoromethyl)phenyl]ethyl]azetidine-1-carbonyl]-4,4a,5,7,8,8a-hexahydropyrido[4,3-b][1,4]oxazin-3-one

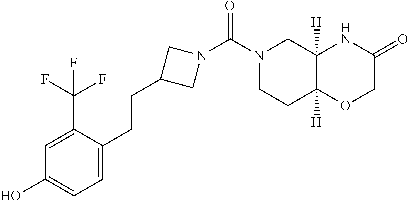 [SMILES: O=C1CO[C@H]2CCN(C(=O)N3CC(CCc4ccc(O)cc4C(F)(F)F)C3)C[C@H]2N1]

(4aR,8aS)-6-[3-[4-Methoxy-2-(trifluoromethyl)phenethyl]azetidine-1-carbonyl]hexahydro-2H-pyrido[4,3-b][1,4]oxazin-3(4H)-one (20 mg, 45.3 μmol) was combined with DCM (0.5 ml). After cooling to 0° C. BBr₃ (11.3 mg, 4.29 μl, 45.3 μmol) was added. The reaction mixture was stirred 3 h at rt. The reaction mixture was quenched by addition of sat. aq. NaHCO₃ and then extracted with EtOAc, dried over Na₂SO₄ and evaporated to dryness. The crude compound was purified by prep. HPLC to afford the title compound (8 mg, 41%) as white solid. MS (ESI): m/z=428.2 [M+H]⁺.

### Example 5

### (4aR,8aS)-6-[3-(4-Cyclobutylphenyl)azetidine-1-carbonyl]-4,4a,5,7,8,8a-hexahydropyrido[4,3-b][1,4]oxazin-3-one

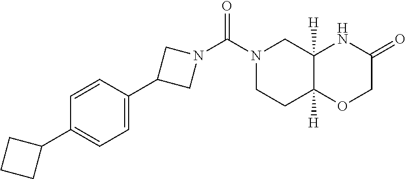 [SMILES: O=C1CO[C@H]2CCN(C(=O)N3CC(c4ccc(C5CCC5)cc4)C3)C[C@H]2N1]

To a vial equipped with a stirring bar was added [Ir{dF(CF₃)ppy}2(dtbpy)]PF₆ (CAS NR 870987-63-6) (1.42 mg, 1.27 μmol), bromocyclobutane (17.1 mg, 127 μmol), (4aR,8aS)-6-[3-(4-bromophenyl)azetidine-1-carbonyl]hexahydro-2H-pyrido[4,3-b][1,4]oxazin-3(4H)-one (50 mg, 127 μmol), tris(trimethylsilyl)silane (31.5 mg, 39.1 μl, 127 μmol) and anhydrous sodium carbonate (26.9 mg, 254 μmol). The vial was sealed and placed under Ar before DME (1 mL) was added. To a separate vial were added dichloronickel 1,2-dimethoxyethane (2.79 mg, 12.7 μmol) and 4-tert-butyl-2-(4-tert-butyl-2-pyridyl)pyridine (3.4 mg, 12.7 μmol). The precatalyst vial was sealed, purged with Ar and DME (4 mL) was added. The precatalyst vial was sonicated for 5 min, after which, 0.4 mL (0.5 mol % catalyst) of it was passed with a syringe into the reaction vessel. The reaction mixture was degassed with Ar. The reaction was stirred and irradiated with a 420 nm lamp for 14.5 h. The reaction was quenched by exposure to air, filtered and washed with a small volume of EtOAc. The filtrate was treated with silica gel and evaporated. The compound was purified by silica gel chromatography on a 12 g column using an MPLC (ISCO) system eluting with a gradient of n-heptane:EtOAc/EtOH 3/1 (80:20 to 10:90) to get the desired compound as a light brown gum (22 mg; 47%). Further purification on a preparative HPLC (YMC-Triart C18 column) using a gradient of ACN:H₂O (0.1% formic acid) (35:65 to 100:0) afforded the desired compound as colorless solid (15 mg, 32%). MS (ESI): m/z=370.3 [M+H]⁺.

Intermediate:

### a) (4aR,8aS)-6-[3-(4-bromophenyl)azetidine-1-carbonyl]hexahydro-2H-pyrido[4,3-b][1,4]oxazin-3(4H)-one

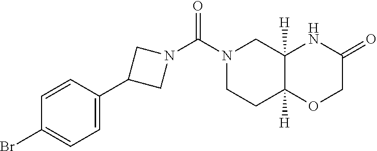 [SMILES: O=C1CO[C@H]2CCN(C(=O)N3CC(c4ccc(Br)cc4)C3)C[C@H]2N1]

To a suspension of 3-(4-bromophenyl)azetidine hydrochloride (CAS NR 90561-74-3) (83.0 mg, 334 μmol) and 4-nitrophenyl (4aR,8aS)-3-oxohexahydro-2H-pyrido[4,3-b][1,4]oxazine-6(5H)-carboxylate (BB2) (107 mg, 334 μmol) in ACN (2 mL) was added DIPEA (173 mg, 233 μL, 1.34 mmol) and the mixture was stirred overnight at RT. The yellow solution was evaporated. The product was purified on a preparative HPLC (Gemini NX column) using a gradient of ACN:H₂O (0.1% TEA) (20:80 to 98:2) to get the desired compound (193 mg, 81%) as colorless gum. MS (ESI): m/z=394.2 [M+H]⁺.

### Example [¹¹C]5

### [¹¹C](4aR,8aS)-6-[3-(4-Cyclobutylphenyl)azetidine-1-carbonyl]-4,4a,5,7,8,8a-hexahydropyrido[4,3-b][1,4]oxazin-3-one

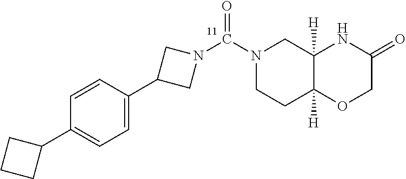 [SMILES: O=C1CO[C@H]2CCN(C(=O)N3CC(c4ccc(C5CCC5)cc4)C3)C[C@H]2N1]

The general procedure described above was applied with (4-cyclobutylphenyl)azetidine 4-methylbenzenesulfonate (4.7 μmol, 0.88 mg) as a precursor at the third step. The product was obtained with a radiochemical purity above 99% and a molar activity of 64-96 GBq/μmol.

Intermediates:

### a) tert-Butyl 3-(4-bromophenyl)azetidine-1-carboxylate

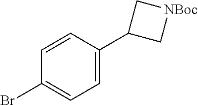 [SMILES: CC(C)(C)OC(=O)N1CC(c2ccc(Br)cc2)C1]

To a stirred solution of tert-butyl 3-iodoazetidine-1-carboxylate (CAS NR 254454-54-1) (1.20 g, 4.24 mmol) in 2-propanol (15 mL) was added (4-bromophenyl)boronic acid (1.70 g, 8.48 mmol) at RT. To the mixture was added rac-(1R,2R)-2-aminocyclohexan-1-ol (29.3 mg, 254 μmol), nickel(II) iodide (79.5 mg, 254 μmol) and sodium bis(trimethylsilyl)amide 2M in THF (4.24 mL, 8.48 mmol) under Ar. The mixture was heated in a microwave oven for 30 min at 80° C. The reaction mixture was poured on water and EtOAc. The aqueous layer was extracted twice with EtOAc. The organic layers were dried over MgSO₄, treated with silica gel and evaporated. The compound was purified by silica gel chromatography first on a 40 g and then two additional times on 80 g columns using an MPLC system eluting with a gradient of n-heptane:EtOAc (100:0 to 50:50) to get the desired compound (440 mg, 33%) as colorless oil. MS (ESI): m/z=256.0 [M-C₄H₈+H]⁺.

### b) tert-Butyl 3-(4-cyclobutylphenyl)azetidine-1-carboxylate

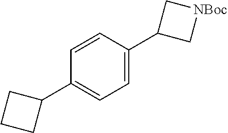 [SMILES: CC(C)(C)OC(=O)N1CC(c2ccc(C3CCC3)cc2)C1]

The title compound was prepared in analogy to (4aR,8aS)-6-[3-(4-cyclobutylphenyl)azetidine-1-carbonyl]-4,4a,5,7,8,8a-hexahydropyrido[4,3-b][1,4]oxazin-3-one (example 5) from bromocyclobutane (381 mg, 2.82 mmol) and tert-butyl 3-(4-bromophenyl)azetidine-1-carboxylate (440 mg, 1.41 mmol). 125 mg (30%) colorless solid. MS (ESI): m/z=232.1 [M-C₄H₈+H]⁺.

### c) 3-(4-Cyclobutylphenyl)azetidine 4-methylbenzenesulfonate

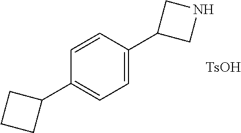

To a mixture of tert-butyl 3-(4-cyclobutylphenyl)azetidine-1-carboxylate (390 mg, 1.36 mmol) in EtOAc (6 mL) was added p-toluenesulfonic acid monohydrate (258 mg, 1.36 mmol) and the mixture was stirred at reflux in a sealed tube for 1 h. The reaction mixture was cooled in a fridge at 4° C. for 30 min, then solids were collected by filtration. The filter cake was washed with a small volume of EtOAc and dried to get the desired compound (377 mg; 73%) as colorless solid. MS (ESI): m/z=188.2 [M+H]⁺.

### Example 6

### (4aR,8aS)-6-[6-[(2,6-Difluorophenyl)methyl]-2-azaspiro[3.3]heptane-2-carbonyl]-4,4a,5,7,8,8a-hexahydropyrido[4,3-b][1,4]oxazin-3-one

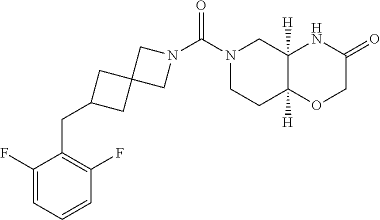 [SMILES: O=C1CO[C@H]2CCN(C(=O)N3CC4(CC(Cc5c(F)cccc5F)C4)C3)C[C@H]2N1]

bis(Trichloromethyl) carbonate (39.9 mg, 134 μmol) and sodium bicarbonate (64.5 mg, 768 μmol) were combined under Ar. DCM (10 ml) was added to give a suspension. (4aR,8aS)-hexahydro-2H-pyrido[4,3-b][1,4]oxazin-3(4H)-one (BB1) (30 mg, 192 μmol) was added to the suspension at 0° C. The mixture was stirred at 0° C. for 5 min, then at RT for 20 h. 6-(2,6-Difluorobenzyl)-2-azaspiro[3.3]heptane 2,2,2-trifluoroacetate (64.8 mg, 192 μmol) and DIPEA (99.3 mg, 134 μl, 768 μmol) were added. The resulting off-white suspension was stirred at RT for 1 h. The reaction mixture was poured into H₂O (20 mL) and extracted with DCM (2×50 mL). The organic layers were combined, washed with brine, dried over Na₂SO₄ and concentrated under vacuum. The crude material was purified by flash chromatography (silica gel, 20 g, 0% to 10% MeOH in DCM). 52 mg (65%), white foam. MS (ESI): m/z=406.2 [M+H]⁺.

Intermediates:

### a) tert-butyl 6-(2,6-difluorobenzylidene)-2-azaspiro[3.3]heptane-2-carboxylate

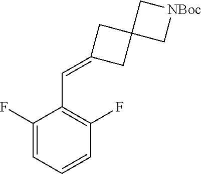 [SMILES: CC(C)(C)OC(=O)N1CC2(CC(=Cc3c(F)cccc3F)C2)C1]

Under Ar at −78° C., (2,6-difluorobenzyl)triphenylphosphonium bromide (CAS NR 159783-80-9) (0.5 g, 1.07 mmol) was dissolved in dry THF (5 ml) and LHMDS (2.13 ml, 2.13 mmol) was added. The reaction mixture was stirred at −78° C. for 2 h. Then at RT, tert-butyl 6-oxo-2-azaspiro[3.3]heptane-2-carboxylate (CAS NR 1181816-12-5) (225 mg, 1.07 mmol) was added and the mixture was stirred at 85° C. overnight. TBDME was added and precipitating TPPO was filtered off. The filtrate was concentrated and purified by flash chromatography (silica gel, 20 g, 0% to 80% EtOAc in heptane). 114 mg (33%), white solid. MS (ESI): m/z=266.2 [M-C₄H₈+H]⁺.

### b) tert-Butyl 6-(2,6-difluorobenzyl)-2-azaspiro[3.3]heptane-2-carboxylate

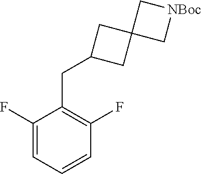 [SMILES: CC(C)(C)OC(=O)N1CC2(CC(Cc3c(F)cccc3F)C2)C1]

tert-Butyl 6-(2,6-difluorobenzylidene)-2-azaspiro[3.3]heptane-2-carboxylate (0.114 g, 355 μmol) was dissolved in EtOAc (2 ml). The flask was purged and backfilled with Ar, Pd/C 10% (37.8 mg, 35.5 μmol) was added and the reaction was stirred under H₂ for 2 h. The reaction mixture was filtered through a Celite pad, wash with EtOAc and dried on the HV. 112 mg (98%), colorless oil. MS (ESI): m/z=268.2 [M-C₄H₈+H]⁺.

### c) 6-(2,6-Difluorobenzyl)-2-azaspiro[3.3]heptane 2,2,2-trifluoroacetate

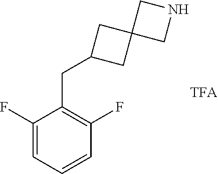 [SMILES: Fc1cccc(F)c1CC1CC2(CNC2)C1]

To a solution of tert-butyl 6-(2,6-difluorobenzyl)-2-azaspiro[3.3]heptane-2-carboxylate (112 mg, 346 μmol) in DCM (3 ml) was added 2,2,2-trifluoroacetic acid (197 mg, 1.73 mmol). The resulting reaction mixture was stirred at RT for 2 h before evaporation of volatiles on the HV (wash with Top. 124 mg (quant.), colorless oil. MS (ESI): m/z=224.1 [M+H]⁺.

### Example [¹¹C]6

### [¹¹C](4aR,8aS)-6-[6-[(2,6-Difluorophenyl)methyl]-2-azaspiro[3.3]heptane-2-carbonyl]-4,4a,5,7,8,8a-hexahydropyrido[4,3-b][1,4]oxazin-3-one

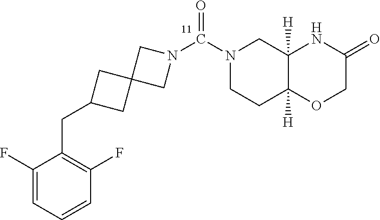 [SMILES: O=C1CO[C@H]2CCN(C(=O)N3CC4(CC(Cc5c(F)cccc5F)C4)C3)C[C@H]2N1]

The general procedure described above was applied with 6-(2,6-difluorobenzyl)-2-azaspiro[3.3]heptane 2,2,2-trifluoroacetate (4.7 μmol, 1.05 mg) as a precursor at the third step. The product was obtained with a radiochemical purity above 99%.

### Example 7

### (4aR,8aS)-6-[6-[(2-Methoxyphenyl)methyl]-2-azaspiro[3.3]heptane-2-carbonyl]-4,4a,5,7,8,8a-hexahydropyrido[4,3-b][1,4]oxazin-3-one

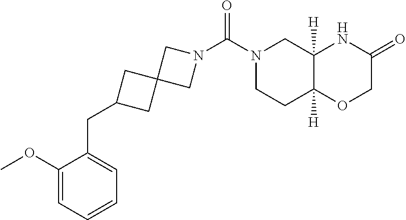 [SMILES: COc1ccccc1CC1CC2(C1)CN(C(=O)N1CC[C@@H]3OCC(=O)N[C@@H]3C1)C2]

(4aR,8aS)-4a,5,6,7,8,8a-Hexahydro-4H-pyrido[4,3-b][1,4]oxazin-6-ium-3-one; (2S,3S)-2,3-bis[(4-methylbenzoyl)oxy]butanedioic acid; (2S,3S)-4-hydroxy-2,3-bis[(4-methylbenzoyl)oxy]-4-oxobutanoate; hydrate (salt of BB1) (100 mg, 144 μmol) was suspended in ACN (2 mL) and TEA was added (102 mg, 140 μl, 1 mmol) under stirring at RT. Then bis(1,2,4-triazol-1-yl)methanone (23.6 mg, 144 μmol) was added in one portion. The reaction mixture was stirred at RT for 2 h. 6-(2-Methoxybenzyl)-2-azaspiro[3.3]heptane 2,2,2-trifluoroacetate (57.1 mg, 172 μmol) dissolved in ACN (100 μl) was added dropwise at RT. The reaction mixture was heated at 50° C. over 10 min and stirred at that temperature for 3 h. The reaction mixture was cooled down to r.t, quenched with water (2 mL), and then extracted with TBME (4 mL). The organic layer was washed with 5% aq. NaHCO₃ (2 mL), then with 0.5 M HCl (1 mL) and brine. After drying over Na₂SO₄ and evaporation of the solvent the residue was purified by flash chromatography (silica gel, 20 g, 0% to 10% MeOH in DCM). 52 mg (91%), white foam. MS (ESI): m/z=400.2 [M+H]⁺.

Intermediates:

### a) tert-Butyl 6-(2-methoxybenzylidene)-2-azaspiro[3.3]heptane-2-carboxylate

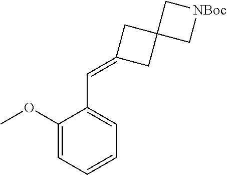 [SMILES: COc1ccccc1C=C1CC2(C1)CN(C(=O)OC(C)(C)C)C2]

Under Ar at −78° C., (2-methoxybenzyl)triphenylphosphonium bromide (CAS NR 64820-07-1) (0.5 g, 1.08 mmol) was dissolved in dry THF (10 ml) and LHMDS (2.16 ml, 2.16 mmol) was added. The reaction mixture was stirred at −78° C. for 2 h. Then at RT, tert-butyl 6-oxo-2-azaspiro[3.3]heptane-2-carboxylate (CAS NR 1181816-12-5) (228 mg, 1.08 mmol) was added and the mixture was stirred at 85° C. overnight. TBDME was added and precipitated TPPO was filtrated off. The filtrate was concentrated on directly purified by flash chromatography (silica gel, 20 g, 0% to 80% EtOAc in heptane). 166 mg (49%), yellow solid. MS (ESI): m/z=260.2 [M-C₄H₈+H]⁺.

### b) tert-Butyl 6-(2-methoxybenzyl)-2-azaspiro[3.3]heptane-2-carboxylate

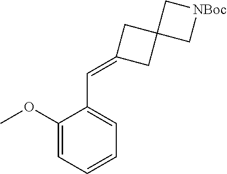 [SMILES: COc1ccccc1C=C1CC2(C1)CN(C(=O)OC(C)(C)C)C2]

tert-Butyl 6-(2-methoxybenzylidene)-2-azaspiro[3.3]heptane-2-carboxylate (166 mg, 526 μmol) was dissolved in EtOAc (3 ml). The flask was purged and backfilled with Ar (×3). Pd/C 10% (56 mg, 52.6 μmol) was added and the reaction was stirred under H₂ for 2 h. The mixture was filtered through a Celite pad, washed with EtOAc and dried on the HV. 120 mg (72%), colorless oil. MS (ESI): m/z=262.2 [M-C₄H₈+H]⁺.

### c) 6-(2-Methoxybenzyl)-2-azaspiro[3.3]heptane 2,2,2-trifluoroacetate

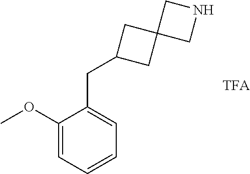 [SMILES: COc1ccccc1CC1CC2(CNC2)C1]

To a solution of tert-butyl 6-(2-methoxybenzyl)-2-azaspiro[3.3]heptane-2-carboxylate (120 mg, 378 μmol) in DCM (3 ml) was added 2,2,2-trifluoroacetic acid (216 mg, 145 μl, 1.89 mmol). The resultant reaction mixture was stirred at RT for 2 h. The reaction mixture was concentrated under HV (washed with Tol). 125 mg (100%), colorless oil. MS (ESI): m/z=218.1 [M+H]⁺.

### Example [³H]7

### (4aR,8aS)-6-[6-[(2-Methoxyphenyl)methyl]-2-azaspiro[3.3]heptane-2-carbonyl]-4,4a,5,7,8,8a-hexahydropyrido[4,3-b][1,4]oxazin-3-one RO 7425945-000-001

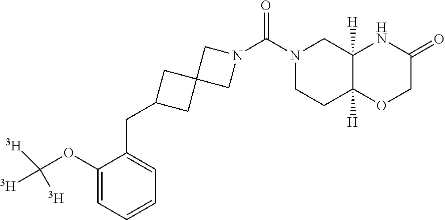 [SMILES: [3H]C([3H])([3H])Oc1ccccc1CC1CC2(C1)CN(C(=O)N1CC[C@@H]3OCC(=O)N[C@@H]3C1)C2]

To a solution of [³H]methyl nosylate (1.85 GBq, 50 mCi, 0.625 μmol) in DMF (100 μl) the phenol precursor (4aR,8aS)-6-(6-(2-hydroxybenzyl)-2-azaspiro[3.3]heptane-2-carbonyl)hexahydro-2H-pyrido[4,3-b][1,4]oxazin-3(4H)-one (0.48 mg, 1.25 μmol) dissolved in DMF (150 μl) was added. Cesium carbonate (1 mg, 3.13 μmol) was added and the reaction mixture was stirred for 2 h at r.t. before it was treated with H₂O and TBME. After separation of the organic layer the aqueous layer was again extracted with TBME. The combined organic layers were consecutively washed with 1 N NaOH and water before they were dried over sodium sulfate. After evaporation of the organic solvent the crude product was purified by HPLC (X-Terra Prep RP-18, 10×150 mm, ACN/H₂O (containing 5% of ACN), 3 ml/min, 210 nm). The pure tritium-labeled compound was isolated by solid phase extraction (Sep-Pak Plus C18) and eluted from the cartridge as ethanolic solution to yield 185 MBq (5.0 mCi) of the target compound in 99.5% radiochemical purity and a specific activity of 3.06 TBq/mmol (82.8 Ci/mmol) as determined by mass spectrometry (MS). The identity of the labeled compound was confirmed by HPLC (by co-injecting the unlabeled reference standard) and by MS. MS: m/z=400 [M(H)+H]⁺ (3%), 402 [M(³H)+H]⁺ (0%), 404 [M(³H₂)+H]⁺ (4%), 406 [M(³H₃)+H]⁺ (93%).

Intermediates:

### a) 2-((2-Azaspiro[3.3]heptan-6-yl)methyl)phenol

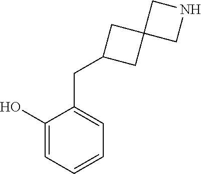 [SMILES: Oc1ccccc1CC1CC2(CNC2)C1]

tert-Butyl 6-(2-methoxybenzyl)-2-azaspiro[3.3]heptane-2-carboxylate (102 mg, 321 μmol) was dissolved in DCM (2 ml). BBr₃ (161 mg, 60.8 μl, 643 μmol) was added at 0° C. The reaction was stirred at RT overnight before quenching by addition of sat. aq. NaHCO₃ and extraction with EtOAc/THF. Organic layers were combined, washed with brine, dried over Na₂SO₄ and concentrated under vacuum. 65 mg (99%), brown oil. MS (ESI): m/z=204.2 [M+H]⁺. Used without further purification in the next step.

### b) (4aR,8aS)-6-[6-[(2-Hydroxyphenyl)methyl]-2-azaspiro[3.3]heptane-2-carbonyl]-4,4a,5,7,8,8a-hexahydropyrido[4,3-b][1,4]oxazin-3-one

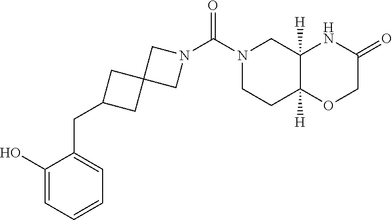 [SMILES: O=C1CO[C@H]2CCN(C(=O)N3CC4(CC(Cc5ccccc5O)C4)C3)C[C@H]2N1]

(4aR,8aS)-4a,5,6,7,8,8a-Hexahydro-4H-pyrido[4,3-b][1,4]oxazin-6-ium-3-one; (2S,3S)-2,3-bis[(4-methylbenzoyl)oxy]butanedioic acid; (2S,3S)-4-hydroxy-2,3-bis[(4-methylbenzoyl)oxy]-4-oxobutanoate; hydrate (salt of BB1) (175 mg, 251 μmol) was suspended in ACN (4 ml) and TEA (178 mg, 245 μl, 1.76 mmol) was added at RT. Then bis(1,2,4-triazol-1-yl)methanone (41.2 mg, 251 μmol) was added in one portion. The reaction mixture was stirred at RT for 2 h. 2-((2-Azaspiro[3.3]heptan-6-yl)methyl)phenol (61.3 mg, 301 μmol) dissolved in ACN (200 μl) was added dropwise at RT. The reaction mixture was heated over 10 min at 50° C. and stirred at that temperature for 3 h. After cooling to RT, water (2 mL) was added and the product was extracted with TBME (4 mL). The organic layer was washed with 5% aq. NaHCO₃ (2 mL), then with 0.5 M HCl (1 mL) and brine. After drying over Na₂SO₄ and evaporation of the solvent the crude material was purified by flash chromatography (silica gel, 20 g, 0% to 10% MeOH in DCM) to give the title compound. 48 mg (47%, 95% purity), white solid. MS (ESI): m/z=386.2 [M+H]⁺.

### Example 8

### (4aR,8aS)-6-[3-[4-(Cyclopentoxy)phenyl]azetidine-1-carbonyl]-4,4a,5,7,8,8a-hexahydropyrido[4,3-b][1,4]oxazin-3-one

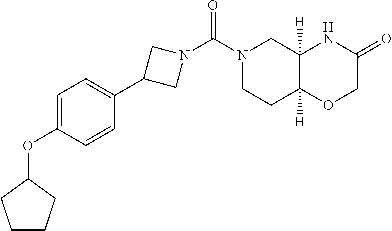 [SMILES: O=C1CO[C@H]2CCN(C(=O)N3CC(c4ccc(OC5CCCC5)cc4)C3)C[C@H]2N1]

tert-Butyl 3-(4-(cyclopentyloxy)phenyl)azetidine-1-carboxylate (150 mg, 473 μmol) was dissolves in 1,1,1,3,3,3-hexafluoropropan-2-ol (2 ml) and stirred for 40 minutes at 150° C. in the microwave. Then the solution was completely evaporated. The crude intermediate was suspended in ACN (1.5 ml), 4-nitrophenyl (4aR,8aS)-3-oxohexahydro-2H-pyrido[4,3-b][1,4]oxazine-6(5H)-carboxylate (BB2) (152 mg, 473 μmol) and DIPEA (244 mg, 330 μl, 1.89 mmol) were added and the mixture was stirred at RT over night. The suspension was completely evaporated. The product was purified on a preparative HPLC (YMC-Triart column) using a gradient of ACN:H₂O (0.1% TEA) (20:80 to 40:60 to 55:45 to 0:100) to get the desired compound as colorless solid (32 mg; 17%). MS (ESI): m/z=400.3 [M+H]⁺.

Intermediate:

### a) tert-Butyl 3-(4-(cyclopentyloxy)phenyl)azetidine-1-carboxylate

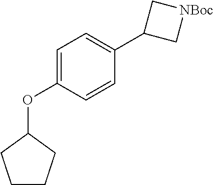 [SMILES: CC(C)(C)OC(=O)N1CC(c2ccc(OC3CCCC3)cc2)C1]

The title compound was prepared in analogy to (4aR,8aS)-6-[3-(4-cyclobutylphenyl)azetidine-1-carbonyl]-4,4a,5,7,8,8a-hexahydropyrido[4,3-b][1,4]oxazin-3-one (example 5) from 1-bromo-4-(cyclopentyloxy)benzene (CAS NR 30752-30-8) (400 mg, 1.66 mmol) and tert-butyl 3-(4-bromophenyl)azetidine-1-carboxylate (588 mg, 2.49 mmol). 444 mg (84%), colorless oil. MS (ESI): m/z=262.1 [M+H]⁺.

### Example 9

### (4aR,8aS)-6-[3-(4-isobutoxyphenyl)azetidine-1-carbonyl]-4,4a,5,7,8,8a-hexahydropyrido[4,3-b][1,4]oxazin-3-one

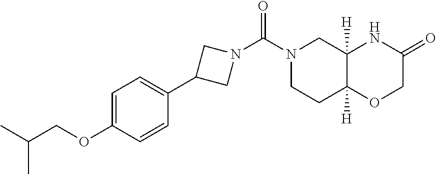 [SMILES: CC(C)COc1ccc(C2CN(C(=O)N3CC[C@@H]4OCC(=O)N[C@@H]4C3)C2)cc1]

To a suspension of (4aR,8aS)-6-(3-(4-hydroxyphenyl)azetidine-1-carbonyl)hexahydro-2H-pyrido[4,3-b][1,4]oxazin-3(4H)-one (0.056 g, 169 μmol) and potassium carbonate (28 mg, 203 μmol) in DMF (0.7 mL) was added 1-iodo-2-methylpropane (37.3 mg, 23.3 μL, 203 μmol) and the mixture was stirred at RT overnight, then at 50° C. for 3 h. More 1-iodo-2-methylpropane (37.3 mg, 23.3 μL, 203 μmol) and potassium carbonate (28 mg, 203 μmol) were added and stirring was continued at 50° C. overnight. A third portion of 1-iodo-2-methylpropane (62.2 mg, 38.9 μL, 338 μmol) was added and stirring was continued at 50° C. for additional 2 h. The mixture was filtered and the filter cake was washed with a few drops of DMF. The product was purified on a preparative HPLC (Gemini NX column) using a gradient of ACN:H₂O (0.1% HCOOH) (20:80 to 100:0) to get the desired compound as colorless solid (8 mg; 12%). MS (ESI): m/z=388.2 [M+H]⁺.

Intermediates:

### a) tert-Butyl 3-(4-(tert-butoxy)phenyl)azetidine-1-carboxylate

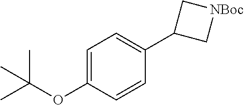 [SMILES: CC(C)(C)OC(=O)N1CC(c2ccc(OC(C)(C)C)cc2)C1]

The title compound was prepared in analogy to (4aR,8aS)-6-[3-(4-cyclobutylphenyl)azetidine-1-carbonyl]-4,4a,5,7,8,8a-hexahydropyrido[4,3-b][1,4]oxazin-3-one (example 5) from 1-bromo-4-(tert-butoxy)benzene (CAS NR 60876-70-2) (400 mg, 1.75 mmol) and tert-butyl 3-(4-bromophenyl)azetidine-1-carboxylate (618 mg, 2.62 mmol). 241 mg (41%, 90% purity), colorless solid. MS (ESI): m/z=250.2 [M-C₄H₈+H]⁺.

### b) (4aR,8aS)-6-(3-(4-Hydroxyphenyl)azetidine-1-carbonyl)hexahydro-2H-pyrido[4,3-b][1,4]oxazin-3(4H)-one

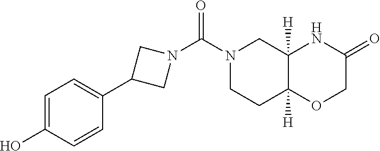 [SMILES: O=C1CO[C@H]2CCN(C(=O)N3CC(c4ccc(O)cc4)C3)C[C@H]2N1]

A solution of tert-butyl 3-(4-(tert-butoxy)phenyl)azetidine-1-carboxylate (161 mg, 527 μmol) and 4-methylbenzenesulfonic acid hydrate (100 mg, 527 μmol) in EtOAc (1 ml) was stirred at RT for 5 h. More 4-methylbenzenesulfonic acid hydrate (20.1 mg, 105 μmol) was added and the reaction was stirred for 30 min at RT. DIPEA (273 mg, 368 μL, 2.11 mmol) was added and the suspension was completely evaporated. The intermediate was suspended in ACN (1 ml) and 4-nitrophenyl (4aR,8aS)-3-oxohexahydro-2H-pyrido[4,3-b][1,4]oxazine-6(5H)-carboxylate (BB2) (199 mg, 527 μmol) was added and the mixture was stirred at RT over night. The suspension was completely evaporated. The product was purified on a preparative HPLC (Gemini NX column) using a gradient of ACN (10-70-100%) in H₂O (0.1% TEA) to get the desired compound as colorless solid (49 mg; 24%). MS (ESI): m/z=388.3 [M+H]⁺.

### Examples 10, 11 and 12

### (4aR,8aS)-6-[3-[2-Fluoro-2-(3-fluoro-4-methyl-phenyl)vinyl]azetidine-1-carbonyl]-4,4a,5,7,8,8a-hexahydropyrido[4,3-b][1,4]oxazin-3-one (10)

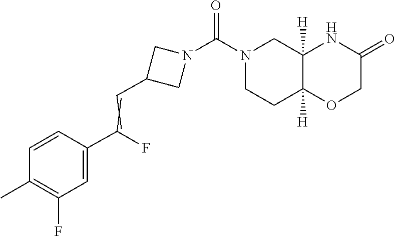 [SMILES: Cc1ccc(C(F)=CC2CN(C(=O)N3CC[C@@H]4OCC(=O)N[C@@H]4C3)C2)cc1F]

### (4aR,8aS)-6-[3-[(Z)-2-Fluoro-2-(3-fluoro-4-methyl-phenyl)vinyl]azetidine-1-carbonyl]-4,4a,5,7,8,8a-hexahydropyrido[4,3-b][1,4]oxazin-3-one (11)

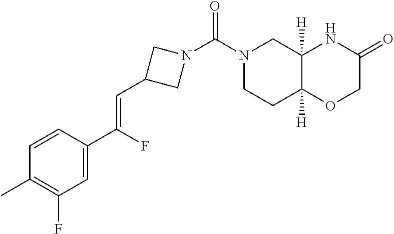 [SMILES: Cc1ccc(/C(F)=C/C2CN(C(=O)N3CC[C@@H]4OCC(=O)N[C@@H]4C3)C2)cc1F]

### (4aR,8aS)-6-[3-[(E)-2-Fluoro-2-(3-fluoro-4-methyl-phenyl)vinyl]azetidine-1-carbonyl]-4,4a,5,7,8,8a-hexahydropyrido[4,3-b][1,4]oxazin-3-one (12)

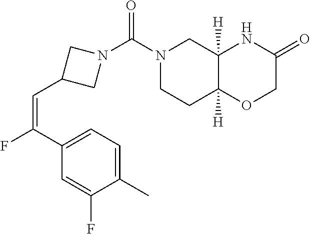 [SMILES: Cc1ccc(/C(F)=C\C2CN(C(=O)N3CC[C@@H]4OCC(=O)N[C@@H]4C3)C2)cc1F]

Triphosgene (14 mg, 0.048 mmol) was dissolved in CH₂Cl₂ (2 mL). A mixture of (4aR,8aS)-4a,5,6,7,8,8a-hexahydro-4H-pyrido[4,3-b][1,4]oxazin-3-one (BB1) (20 mg, 0.129 mmol) and diisopropylethylamine (37 mg, 0.284 mmol) in DMF (5 mL) was slowly added to the stirred solution of triphosgene. After a further 5 min of stirring, a solution of 3-(2-fluoro-2-(3-fluoro-4-methylphenyl)vinyl)azetidine (27 mg, 0.129 mmol) and diisopropylethylamine (37 mg, 0.284 mmol) in CH₂Cl₂ (2 mL) was added in one portion. After stirring at room temperature overnight, the mixture was evaporated to dryness and re-dissolved in EtOAc, washed with aq. sat. NaHCO₃ and brine and dried over MgSO₄. After filtration and evaporation, the residue was purified by flash column chromatography (silica gel, 1:10 (v/v) MeOH/DCM) to afford (4aR,8aS)-6-[3-[2-fluoro-2-(3-fluoro-4-methyl-phenyl)vinyl]azetidine-1-carbonyl]-4,4a,5,7,8,8a-hexahydropyrido[4,3-b][1,4]oxazin-3-one as a light yellow oil (18 mg, 37%). HRMS (ESI) calculated for C₂₀H₂₃F₂N₃NaO₃⁺ [M+Na]⁺ 414.1600 m/z, found 414.1597 m/z.

The (E)-isomer and (Z)-isomer were obtained in small quantities by semi-preparative HPLC.

(4aR,8aS)-6-[3-[(E)-2-Fluoro-2-(3-fluoro-4-methyl-phenyl)vinyl]azetidine-1-carbonyl]-4,4a,5,7,8,8a-hexahydropyrido[4,3-b][1,4]oxazin-3-one: white powder; ¹H NMR (400 MHz, CDCl₃) δ 7.24 (t, J=7.9 Hz, 1H), 7.07-6.96 (m, 2H), 6.80 (s, 1H, CONH—), 5.65 (dd, J=19.9, 9.7 Hz, 1H), 4.42-4.17 (m, 4H), 4.04-3.80 (m, 4H), 3.61-3.49 (m, 2H), 3.48-3.40 (m, 1H), 3.16-3.05 (m, 2H), 2.33 (s, 3H), 2.01-1.93 (m, 1H), 1.89-1.77 (m, 1H)

(4aR,8aS)-6-[3-[(Z)-2-Fluoro-2-(3-fluoro-4-methyl-phenyl)vinyl]azetidine-1-carbonyl]-4,4a,5,7,8,8a-hexahydropyrido[4,3-b][1,4]oxazin-3-one: colorless oil; ¹H NMR (400 MHz, CDCl₃)δ 7.24-7.10 (m, 3H), 6.67 (s, 1H, CONH—), 5.60 (dd, J=35.8, 8.5 Hz, 1H), 4.40-4.20 (m, 4H), 4.05-3.75 (m, 5H), 3.60-3.41 (m, 2H), 3.20-3.05 (m, 2H), 2.31 (d, J=2.0 Hz, 3H), 2.01-1.94 (m, 1H), 1.90-1.79 (m, 1H). HRMS (ESI) calculated for C₂₀H₂₃F₂N₃NaO₃⁺ [M+Na]⁺ 414.1600 m/z, found 414.1597 m/z.

Intermediates:

### a) Diethoxyphosphoryl-(3-fluoro-4-methyl-phenyl)methanol

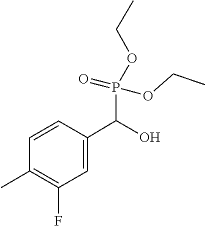 [SMILES: CCOP(=O)(OCC)C(O)c1ccc(C)c(F)c1]

A mixture of 3-fluoro-4-methylbenzaldehyde (3.00 g, 21.72 mmol), diethyl phosphite (6.00 g, 43.43 mmol) and triethylamine (88 mg, 0.87 mmol) was stirred at 50° C. overnight. The resulting precipitate was isolated by filtration, and subsequently washed with Et₂O to afford the title compound as white powder (4605 mg, 77%). ¹H NMR (400 MHz, CDCl₃) δ 7.24-7.14 (m, 3H), 5.00 (d, J=10.6 Hz, 1H), 4.21-3.97 (m, 4H), 2.31-2.28 (m, 3H), 1.44-1.13 (m, 6H). ³¹P NMR (162 MHz, CDCl₃) δ 20.81. HRMS (ESI) calculated for C₁₂H₁₈FNaO₄P⁺ [M+Na]⁺ 299.0819 m/z, found 299.0818 m/z.

### b) 4-[Diethoxyphosphoryl(fluoro)methyl]-2-fluoro-1-methyl-benzene

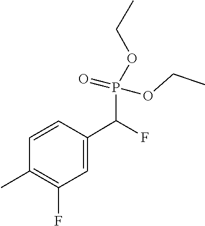 [SMILES: CCOP(=O)(OCC)C(F)c1ccc(C)c(F)c1]

To a solution of diethoxyphosphoryl-(3-fluoro-4-methyl-phenyl)methanol (1.00 g, 3.62 mmol) in DCM (20 mL), diethylaminosulfur trifluoride (876 mg, 5.43 mmol) dissolved in DCM (2 mL) was added slowly at 0° C. using a syringe. The mixture was stirred at room temperature under nitrogen atmosphere for 6 h. Sat. aq. Na₂CO₃ solution was poured into the reaction, and the mixture was extracted with DCM for 3 times. The combined organic layers were washed with brine, dried over anhydrous MgSO₄ and concentrated under reduced pressure. The crude was purified by column chromatography (silica gel, 1:2 (v/v) ethyl acetate/hexane) to afford a light yellow oil (513 mg, 51%). ¹H NMR (400 MHz, CDCl₃) δ 7.26-7.21 (m, 1H), 7.20-7.14 (m, 2H), 5.65 (dd, J=44.7, 7.8 Hz, 1H), 4.25-4.04 (m, 4H), 2.33-2.28 (m, 3H), 1.40-1.26 (m, 6H). HRMS (ESI) calculated for C₁₂H₁₇F₂NaO₃P⁺ [M+Na]⁺ 301.0776 m/z, found 301.0774 m/z.

### c) tert-Butyl 3-(2-fluoro-2-(3-fluoro-4-methylphenyl)vinyl)azetidine-1-carboxylate

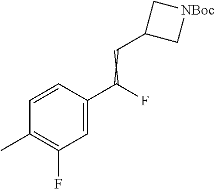 [SMILES: Cc1ccc(C(F)=CC2CN(C(=O)OC(C)(C)C)C2)cc1F]

4-[Diethoxyphosphoryl(fluoro)methyl]-2-fluoro-1-methyl-benzene (100 mg, 0.36 mmol) was dissolved in THF (10 mL) and cooled down to −78° C. Lithium diisopropylamide (50 mg, 0.47 mmol) was added under nitrogen, and the reaction was stirred at −78° C. for 30 min. 1-Boc-azetidine-3-carboxaldehyde (87 mg, 0.47 mmol) was added drop by drop at −78° C., and the mixture was slowly warmed to RT and stirred overnight. H₂O was poured to quench the reaction, and the aqueous layer was extracted with EtOAc. The combined organic layers were dried over anhydrous MgSO₄, filtered, concentrated, and purified by flash column chromatography (silica gel, 1:10 (v/v) ethyl acetate/hexane) to afford a yellow oil (44.3 mg, 40%). ¹H NMR (400 MHz, CDCl₃) δ 7.26-7.13 (m, 2H), 7.04-6.98 (m, 1H), 5.71-5.54 (m, 1H), 4.23 and 4.18 (t, J=8.5 Hz, 2H), 3.85-3.73 (m, 2H), 3.52-3.39 (m, 1H), 2.36-2.27 (m, 3H), 1.48 and 1.46 (s, 9H). HRMS (ESI) calculated for C₁₇H₂₁F₂NNaO₂⁺ [M+Na]⁺ 332.1433 m/z, found 332.1433 m/z.

### d) 3-(2-Fluoro-2-(3-fluoro-4-methylphenyl)vinyl)azetidine

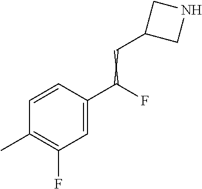

tert-Butyl 3-(2-fluoro-2-(3-fluoro-4-methylphenyl)vinyl)azetidine-1-carboxylate (87 mg, 0.28 mmol) was dissolved at RT in 2 mL DCM and treated with TFA (386 mg, 3.39 mmol). Upon consumption of the starting material, the reaction was neutralized with sat. aq. Na₂CO₃ solution, extracted with DCM, dried over MgSO₄, and concentrated under vacuum. The crude was applied directly in the next step without further purification.

### Example [¹¹C]10

### [¹¹C](4aR,8aS)-6-[3-[2-Fluoro-2-(3-fluoro-4-methyl-phenyl)vinyl]azetidine-1-carbonyl]-4,4a,5,7,8,8a-hexahydropyrido[4,3-b][1, 4]oxazin-3-one

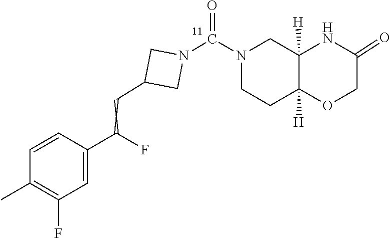 [SMILES: Cc1ccc(C(F)=CC2CN(C(=O)N3CC[C@@H]4OCC(=O)N[C@@H]4C3)C2)cc1F]

The general procedure described above was applied with 3-(2-fluoro-2-(3-fluoro-4-methylphenyl)vinyl)azetidine (4.7 μmol, 0.98 mg) as a precursor at the third step. The product was obtained with a radiochemical purity above 99% and a molar activity of 39-53 GBq/μmol

### MAGL Inhibitory Activity

Compounds were profiled for MAGL inhibitory activity by determining the enzymatic activity by following the hydrolysis of the natural substrate, 2-arachidonoylglycerol, resulting in arachidonic acid, which can be followed by mass spectrometry. This assay is hereinafter abbreviated “2-AG assay”.

The 2-AG assay was carried out in 384 well assay plates (PP, Greiner Cat #784201) in a total volume of 20 μL. Compound dilutions were made in 100% DMSO (VWR Chemicals 23500.297) in a polypropylene plate in 3-fold dilution steps to give a final concentration range in the assay from 12.5 μM to 0.8 pM. 0.25 μL compound dilutions (100% DMSO) were added to 9 μL MAGL in assay buffer (50 mM TRIS (GIBCO, 15567-027), 1 mM EDTA (Fluka, 03690-100 ml), 0.01% (v/v) Tween. After shaking, the plate was incubated for 15 min at RT. To start the reaction, 10 μL 2-arachidonoylglycerol in assay buffer was added. The final concentrations in the assay was 50 pM MAGL and 8 μM 2-arachidonoylglyerol. After shaking and 30 min incubation at RT, the reaction was quenched by the addition of 40 μL of acetonitrile containing 4 μM of d8-arachidonic acid. The amount of arachidonic acid was traced by an online SPE system (Agilent Rapidfire) coupled to a triple quadrupole mass spectrometer (Agilent 6460). A C18 SPE cartridge (G9205A) was used in an acetonitrile/water liquid setup. The mass spectrometer was operated in negative electrospray mode following the mass transitions 303.1→259.1 for arachidonic acid and 311.1→267.0 for d8-arachidonic acid. The activity of the compounds was calculated based on the ratio of intensities [arachidonic acid/d8-arachidonic acid].

TABLE 1

| | | | IC₅₀ MAGL |
|---|---|---|---|
| Example | Name | Structure | [μM] |
| 1 | (4aR,8aS)-6-[3-[2-[2-Fluoro- 6- (trifluoromethyl)phenyl]ethyl] azetidine-1-carbonyl]- 4,4a,5,7,8,8a- hexahydropyrido[4,3- b][1,4]oxazin-3-one | 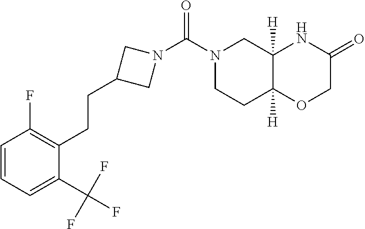 [SMILES: O=C1CO[C@H]2CCN(C(=O)N3CC(CCc4c(F)cccc4C(F)(F)F)C3)C[C@H]2N1] | 0.004 |
| 2 | (4aR,8aS)-6-[6-[(2-Fluoro-6- methoxy-phenyl)methyl]-2- azaspiro[3.3]heptane-2- carbonyl]-4,4a,5,7,8,8a- hexahydropyrido[4,3- b][1,4]oxazin-3-one | 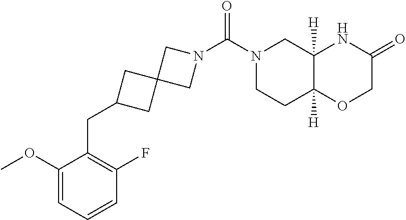 [SMILES: COc1cccc(F)c1CC1CC2(C1)CN(C(=O)N1CC[C@@H]3OCC(=O)N[C@@H]3C1)C2] | 0.00007 |
| 3 | (4aR,8aS)-6-[3-[2-(2-Fluoro- 4-methyl- phenyl)ethyl]azetidine-1- carbonyl]-4,4a,5,7,8,8a- hexahydropyrido[4,3- b][1,4]oxazin-3-one | 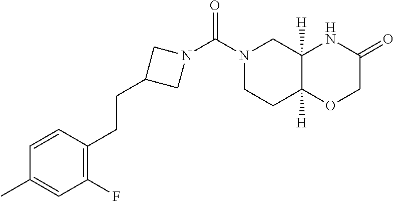 [SMILES: Cc1ccc(CCC2CN(C(=O)N3CC[C@@H]4OCC(=O)N[C@@H]4C3)C2)c(F)c1] | 0.005 |
| 4 | (4aR,8aS)-6-[3-[2-[4- Methoxy-2- (trifluoromethyl)phenyl]ethyl] azetidine-1-carbonyl]- 4,4a,5,7,8,8a- hexahydropyrido[4,3- b][1,4]oxazin-3-one | 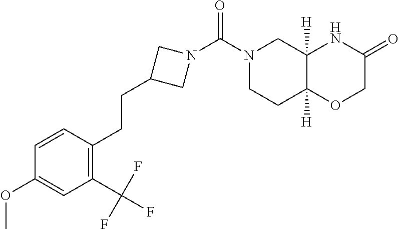 [SMILES: COc1ccc(CCC2CN(C(=O)N3CC[C@@H]4OCC(=O)N[C@@H]4C3)C2)c(C(F)(F)F)c1] | 0.006 |
| 5 | (4aR,8aS)-6-[3-(4- Cyclobutylphenyl)azetidine- 1-carbonyl]-4,4a,5,7,8,8a- hexahydropyrido[4,3- b][1,4]oxazin-3-one | 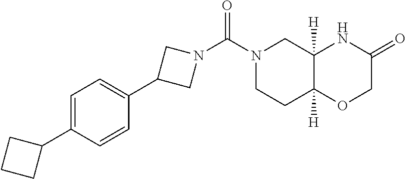 [SMILES: O=C1CO[C@H]2CCN(C(=O)N3CC(c4ccc(C5CCC5)cc4)C3)C[C@H]2N1] | 0.001 |
| 6 | (4aR,8aS)-6-[6-[(2,6- difluorophenyl)methyl]-2- azaspiro[3.3]heptane-2- carbonyl]-4,4a,5,7,8,8a- hexahydropyrido[4,3- b][1,4]oxazin-3-one | 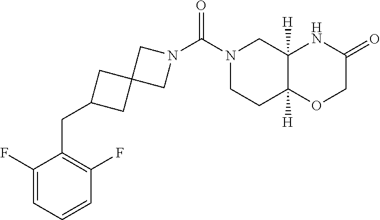 [SMILES: O=C1CO[C@H]2CCN(C(=O)N3CC4(CC(Cc5c(F)cccc5F)C4)C3)C[C@H]2N1] | 0.0001 |
| 7 | (4aR,8aS)-6-[6-[(2- methoxyphenyl)methyl]-2- azaspiro[3.3]heptane-2- carbonyl]-4,4a,5,7,8,8a- hexahydropyrido[4,3- b][1,4]oxazin-3-one | 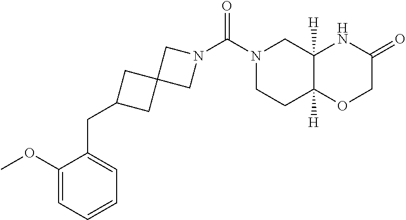 [SMILES: COc1ccccc1CC1CC2(C1)CN(C(=O)N1CC[C@@H]3OCC(=O)N[C@@H]3C1)C2] | 0.0002 |
| 8 | (4aR,8aS)-6-[3-[4- (cyclopentoxy)phenyl] azetidine-1- carbonyl]-4,4a,5,7,8,8a- hexahydropyrido[4,3- b][1,4]oxazin-3-one | 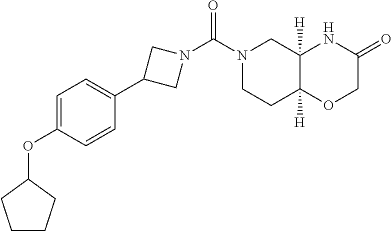 [SMILES: O=C1CO[C@H]2CCN(C(=O)N3CC(c4ccc(OC5CCCC5)cc4)C3)C[C@H]2N1] | 0.0006 |
| 9 | (4aR,8aS)-6-[3-(4- isobutoxyphenyl)azetidine-1- carbonyl]-4,4a,5,7,8,8a- hexahydropyrido[4,3- b][1,4]oxazin-3-one | 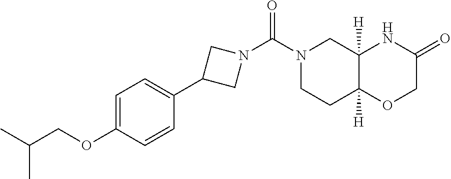 [SMILES: CC(C)COc1ccc(C2CN(C(=O)N3CC[C@@H]4OCC(=O)N[C@@H]4C3)C2)cc1] | 0.001 |
| 10 | (4aR,8aS)-6-[3-[2-fluoro-2- (3-fluoro-4-methyl- phenyl)vinyl]azetidine-1- carbonyl]-4,4a,5,7,8,8a- hexahydropyrido[4,3- b][1,4]oxazin-3-one | 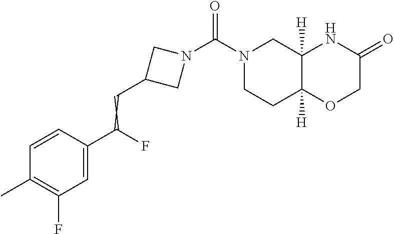 [SMILES: Cc1ccc(C(F)=CC2CN(C(=O)N3CC[C@@H]4OCC(=O)N[C@@H]4C3)C2)cc1F] | 0.02 |
| 11 | (4aR,8aS)-6-[3-[(Z)-2-fluoro- 2-(3-fluoro-4-methyl- phenyl)vinyl]azetidine-1- carbonyl]-4,4a,5,7,8,8a- hexahydropyrido[4,3- b][1,4]oxazin-3-one | 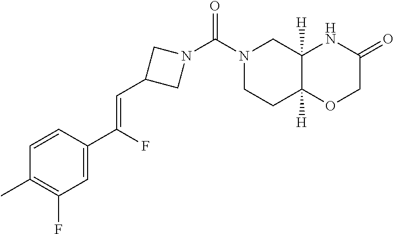 [SMILES: Cc1ccc(/C(F)=C/C2CN(C(=O)N3CC[C@@H]4OCC(=O)N[C@@H]4C3)C2)cc1F] | 0.09 |
| 12 | (4aR,8aS)-6-[3-[(E)-2- fluoro-2-(3-fluoro-4-methyl- phenyl)vinyl]azetidine-1- carbonyl]-4,4a,5,7,8,8a- hexahydropyrido[4,3- b][1,4]oxazin-3-one | 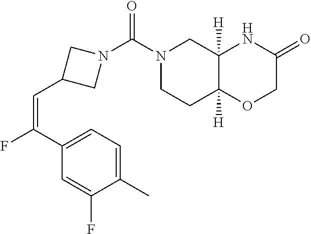 [SMILES: Cc1ccc(/C(F)=C\C2CN(C(=O)N3CC[C@@H]4OCC(=O)N[C@@H]4C3)C2)cc1F] | 0.04 |

### In Vitro Autoradiography of ³H Ligands

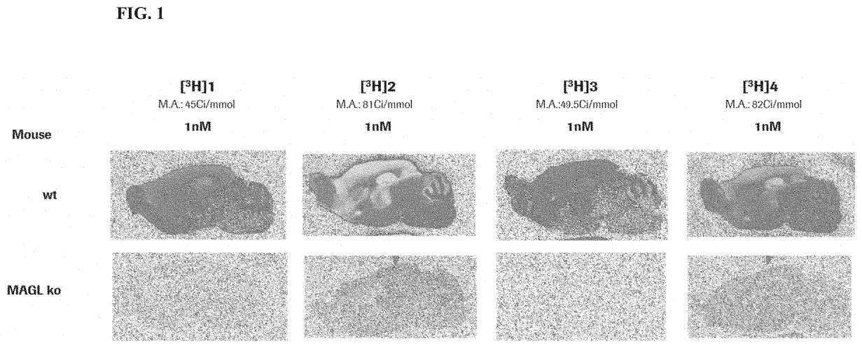

Receptor autoradiography was performed on sagittal sections of fresh-frozen brains from MAGL WT and ko mice (C57BL6/6NTac-Mgllem4993_02Tac). Tissue sections (10 μm) from brain specimens were cut in a cryostat (Leica CM3050) at −17° C. chamber temperature and −15° C. object temperature and thaw-mounted on microscope glass slides (HistoBond, Paul Marienfeld GmbH, Lauda-Königshofen, Germany). Brain sections were incubated for 30 min in 50 mM Tris-HCl buffer pH 7.4 with 0.1% BSA at RT containing 1 nM of the radioligand. Sections were rinsed three times for 10 min in ice-cold 50 mM Tris-HCl buffer, followed by three dips into ice-cold H₂O and air-dried at 4° C. before being exposed to tritium-sensitive imaging plates (BAS-IP TR2025, Fujifilm, Dielsdorf, Switzerland) with a tritium microscale for 5 days at RT. The imaging plates were scanned with a high-resolution phosphor imager (Fuji BAS-5000, Bucher Biotec AG, Basel, Switzerland) and the binding intensity for selected brain areas was quantified using an MCID M2 image analysis software (version 7; InterFocus Imaging GmbH, Mering, Germany). FIG. 1 shows the in vitro autoradiography results of [³H]1, [³H]2, [³H]3 and [³H]4 in sagittal mouse brain sections. Selective binding to MAGL of all four radioligands is observed on wt tissue samples (tow row). Excellent selectivity and low non-specific binding are demonstrated by the absence of binding on MAGL ko tissue sections (bottom row).

### In Vitro Autoradiography of ¹¹C Tracers

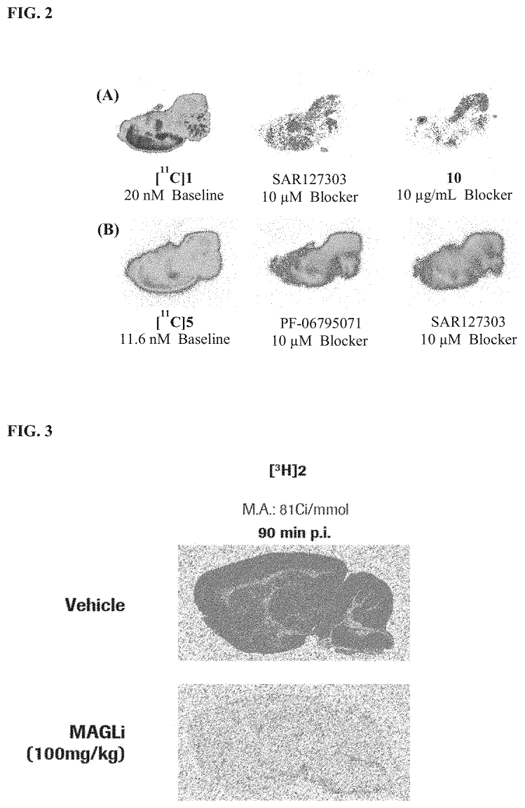

10 μm sagittal brain sections from Wistar rats were used in in vitro autoradiography. On the day of experimentation, the slices were thawed on ice (10 min) and pre-conditioned in an aqueous buffer containing 30 mM 4-(2-hydroxyethyl)-1-piperazineethanesulfonic acid (HEPES), 1.2 mM MgCl₂, 110 mM NaCl, 5 mM KCl, 2.5 mM CaCl₂, and 1% fatty-acid-free bovine serum albumin (pH 7.4, 0° C.) for 10 min. The tissue samples were dried in air and subsequently incubated with radiotracer or a mixture of radiotracer and a cold MAGL inhibitor as a blocker at ambient temperature for 30 min. Upon completion of the incubation time, the slices were washed once for 3 min in the aqueous buffer described above, and twice for 2 min in the aqueous buffer described without 1% fatty-acid-free bovine serum albumin. After two quick dips in distilled water, the sections were dried in the air and exposed to a phosphor image plate for 1 h. The films were scanned by a BAS5000 reader (Fuji), and the data were analyzed using AIDA software, version 4.50.010 (Raytest Isotopenmessgeräte GmbH). For [¹¹C]1, 10 μM SAR127303 and 10 μg/mL 10 were used as blockers. For [¹¹C]5, 10 μM SAR127303 and 10 μM PF-06795071 were used as blockers. FIG. 2 shows the in vitro autoradiography results of [¹¹C]1 (A) and [¹¹C]5 (B) on sagittal rat brain sections. Both radioligands selectively bind to MAGL, and binding can be blocked by co-incubation with high concentrations of cold MAGL inhibitors, thus demonstrating excellent selectivity and specificity.

### In Vivo Binding to MAGL of [³H](4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one

### Example [³H]2

Male CD(SD) rats were pretreated with vehicle or a selective MAGL inhibitor and 60 min later received intravenously 1 mCi/kg of [³H]2 (equivalent to a dose of 5.5 μg/kg). Rats were sacrificed by decapitation 90 min after administration of the radioligand. Brains were rapidly removed and divided in two halves along their sagittal axis. The brain halves were frozen in dry ice for following cryosectioning. The hemisphere was placed in a cryostat and sagittal sections (10 μm thickness) were cut. Brain sections were mounted on Histobond glass slides (Marienfeld Laboratories Glassware, Germany), dried at room temperature and exposed, together with tritium microscales, to tritium-sensitive imaging plates (BAS-TR2025) for five days. The imaging plates were scanned in a Fujifilm BAS-5000 high resolution phosphor imager and the amount of [³H]2 bound to the brain regions of interest was quantified with an MCID M2 image analysis system (Imaging Research Inc., St. Catherines, Ontario, Canada). FIG. 3 shows the ex vivo autoradiography images of sagittal brain sections obtained 90 min post injection of [³H]2. Selective and specific binding to MAGL (top) is blocked by co-administration of a high dose of a MAGL inhibitor (bottom).

### In Vivo PET Scanning in Mice

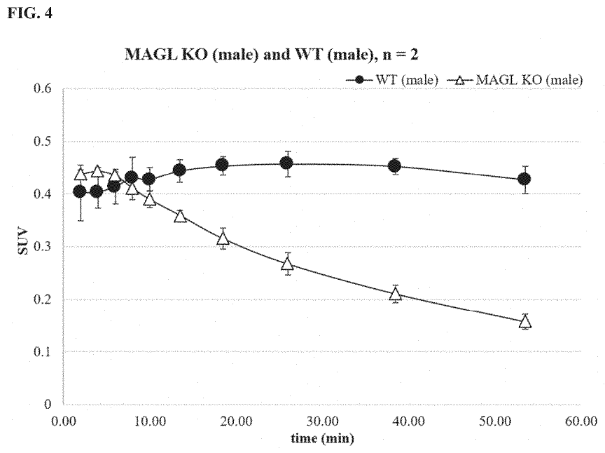

All animals were purchased from Taconic. The animals were anesthetized with isoflurane during the experiment. Dynamic PET scanning started one minute after intravenous injection of [¹¹C]1 (8.7-10.8 MBq), lasted for 1 h, and was followed by CT for anatomic orientation. Time-activity curves of the whole brain were calculated by PMOD, version 4.002 (PMOD Technologies), and presented as SUVs, indicating the decay-corrected radioactivity per cm³ divided by the injected dose per gram of body weight. FIG. 4 shows the time activity curves of the whole brain from [¹¹C]1 in MAGL KO mice and corresponding WT.

1. A radiolabeled compound selected from the group consisting of
(4aR,8aS)-6-[3-[2-[2-fluoro-6-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[3-[2-(2-fluoro-4-methyl-phenyl)ethyl]azetidine-1-carbonyl]-4,4a,5,7,8,8a-hexahydropyrido[4,3-b][1,4]oxazin-3-one;
(4aR,8aS)-6-[3-[2-[4-methoxy-2-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[3-(4-cyclobutylphenyl)azetidine-1-carbonyl]-4,4a,5,7,8,8a-hexahydropyrido[4,3-b][1,4]oxazin-3-one;
(4aR,8aS)-6-[6-[(2,6-difluorophenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[6-[(2-methoxyphenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[3-[4-(cyclopentoxy)phenyl]azetidine-1-carbonyl]-4,4a,5,7,8,8a-hexahydropyrido[4,3-b][1,4]oxazin-3-one;
(4aR,8aS)-6-[3-(4-isobutoxyphenyl)azetidine-1-carbonyl]-4,4a,5,7,8,8a-hexahydropyrido[4,3-b][1,4]oxazin-3-one;
(4aR,8aS)-6-[3-[(Z)-2-fluoro-2-(3-fluoro-4-methyl-phenyl)vinyl]azetidine-1-carbonyl]-4,4a,5,7,8,8a-hexahydropyrido[4,3-b][1,4]oxazin-3-one; and
(4aR,8aS)-6-[3-[(E)-2-fluoro-2-(3-fluoro-4-methyl-phenyl)vinyl]azetidine-1-carbonyl]-4,4a,5,7,8,8a-hexahydropyrido[4,3-b][1,4]oxazin-3-one;
or a pharmaceutically acceptable salt thereof, comprising one or more radioisotopes.

2. The radiolabeled compound comprising one or more radioisotopes according to claim 1, selected from the group consisting of
(4aR,8aS)-6-[3-[2-[2-fluoro-6-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one;
(4aR,8aS)-6-[3-[2-(2-fluoro-4-methyl-phenyl)ethyl]azetidine-1-carbonyl]-4,4a,5,7,8,8a-hexahydropyrido[4,3-b][1,4]oxazin-3-one;
(4aR,8aS)-6-[3-[2-[4-methoxy-2-(trifluoromethyl)phenyl]ethyl]azetidine-1-carbonyl]-4,4a,5,7,8,8a-hexahydropyrido[4,3-b][1,4]oxazin-3-one;
(4aR,8aS)-6-[3-(4-cyclobutylphenyl)azetidine-1-carbonyl]-4,4a,5,7,8,8a-hexahydropyrido[4,3-b][1,4]oxazin-3-one; and
(4aR,8aS)-6-[6-[(2,6-difluorophenyl)methyl]-2-azaspiro[3.3]heptane-2-carbonyl]-4,4a,5,7,8,8a-hexahydropyrido[4,3-b][1,4]oxazin-3-one;
or a pharmaceutically acceptable salt thereof.

3. The radiolabeled compound comprising one or more radioisotopes according to claim 1, selected from the group consisting of
(4aR,8aS)-6-[3-[2-[2-fluoro-6-(trifluoromethyl)phenyl]ethyl]azetidine-1-carbonyl]-4,4a,5,7,8,8a-hexahydropyrido[4,3-b][1,4]oxazin-3-one; and
(4aR,8aS)-6-[6-[(2-fluoro-6-methoxy-phenyl)methyl]-2-azaspiro[3.3]heptane-2-carbonyl]-4,4a,5,7,8,8a-hexahydropyrido[4,3-b][1,4]oxazin-3-one;
or a pharmaceutically acceptable salt thereof.

4. The radiolabeled compound according to any one of claims 1 to 3, or a pharmaceutically acceptable salt thereof, wherein said one or more radioisotopes are imaging isotopes for positron-emission tomography (PET), single-photon emission computed tomography (SPECT) and/or autoradiography.

5. The radiolabeled compound according to any one of claims 1 to 4, or a pharmaceutically acceptable salt thereof, wherein said one or more radioisotopes are independently selected from the group consisting of ³H, ¹¹C, ¹⁴C, ¹³N, ¹⁵O, and ¹⁸F.

6. The radiolabeled compound according to any one of claims 1 to 4, or a pharmaceutically acceptable salt thereof, wherein said one or more radioisotopes are independently selected from the group consisting of ³H, ¹¹C, and ¹⁸F.

7. The radiolabeled compound according to any one of claims 1 to 4, or a pharmaceutically acceptable salt thereof, wherein said one or more radioisotopes are independently selected from the group consisting of ¹¹C and ¹⁸F.

8. The radiolabeled compound according to any one of claims 1 to 7, or a pharmaceutically acceptable salt thereof, comprising 1-4 radioisotopes, e.g. 1, 2, 3 or 4 radioisotopes.

9. The radiolabeled compound according to any one of claims 1 to 7, or a pharmaceutically acceptable salt thereof, comprising 1-3 radioisotopes, e.g. 1, 2 or 3 radioisotopes.

10. The radiolabeled compound according to any one of claims 1 to 7, or a pharmaceutically acceptable salt thereof, comprising 1 radioisotope.

11. The radiolabeled compound comprising one or more radioisotopes according to any one of claims 1 to 10, selected from the group consisting of 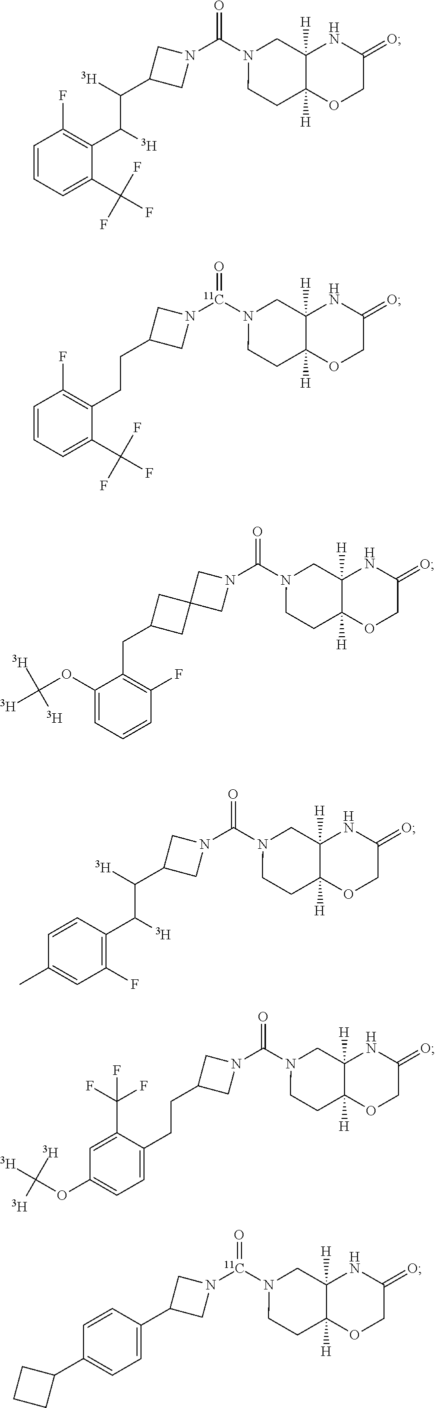 [SMILES: [3H]C(c1c(F)cccc1C(F)(F)F)C([3H])C1CN(C(=O)N2CC[C@@H]3OCC(=O)N[C@@H]3C2)C1 ; O=C1CO[C@H]2CCN([11C](=O)N3CC(CCc4c(F)cccc4C(F)(F)F)C3)C[C@H]2N1 ; [3H]C([3H])([3H])Oc1cccc(F)c1CC1CC2(C1)CN(C(=O)N1CC[C@@H]3OCC(=O)N[C@@H]3C1)C2 ; [3H]C(c1ccc(C)cc1F)C([3H])C1CN(C(=O)N2CC[C@@H]3OCC(=O)N[C@@H]3C2)C1 ; [3H]C([3H])([3H])Oc1ccc(CCC2CN(C(=O)N3CC[C@@H]4OCC(=O)N[C@@H]4C3)C2)c(C(F)(F)F)c1 ; O=C1CO[C@H]2CCN([11C](=O)N3CC(c4ccc(C5CCC5)cc4)C3)C[C@H]2N1] 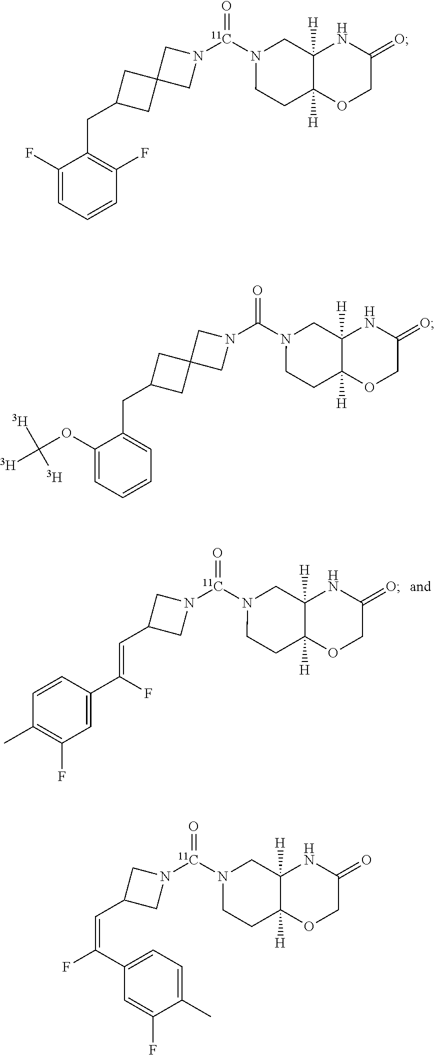 [SMILES: O=C1CO[C@H]2CCN([11C](=O)N3CC4(CC(Cc5c(F)cccc5F)C4)C3)C[C@H]2N1 ; [3H]C([3H])([3H])Oc1ccccc1CC1CC2(C1)CN(C(=O)N1CC[C@@H]3OCC(=O)N[C@@H]3C1)C2 ; Cc1ccc(/C(F)=C/C2CN([11C](=O)N3CC[C@@H]4OCC(=O)N[C@@H]4C3)C2)cc1F ; Cc1ccc(/C(F)=C\C2CN([11C](=O)N3CC[C@@H]4OCC(=O)N[C@@H]4C3)C2)cc1F]
or a pharmaceutically acceptable salt thereof.

12. The radiolabeled compound comprising one or more radioisotopes according to any one of claims 1 to 10, selected from the group consisting of 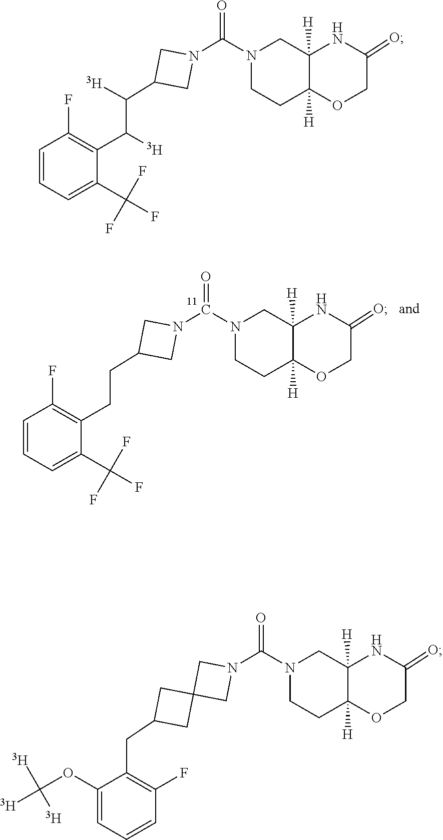 [SMILES: [3H]C(c1c(F)cccc1C(F)(F)F)C([3H])C1CN(C(=O)N2CC[C@@H]3OCC(=O)N[C@@H]3C2)C1 ; O=C1CO[C@H]2CCN(C(=O)N3CC(CCc4c(F)cccc4C(F)(F)F)C3)C[C@H]2N1 ; "and" ; [3H]C([3H])([3H])Oc1cccc(F)c1CC1CC2(C1)CN(C(=O)N1CC[C@@H]3OCC(=O)N[C@@H]3C1)C2]
or a pharmaceutically acceptable salt thereof.

13. The radiolabeled compound comprising one or more radioisotopes according to any one of claims 1 to 12 for use in monoacylglycerol lipase (MAGL) occupancy studies.

14. The radiolabeled compound comprising one or more radioisotopes according to any one of claims 1 to 12 for use in diagnostic imaging of monoacylglycerol lipase (MAGL) in the brain of a mammal.

15. A pharmaceutical composition comprising a radiolabeled compound comprising one or more radioisotopes according to any one of claims 1 to 12 and a pharmaceutically acceptable excipient.

16. A method of diagnostic imaging of monoacylglycerol lipase (MAGL) in the brain of a mammal, comprising:
(a) administering to the mammal a detectable quantity of a radiolabeled compound according to any one of claims 1-12 or of a pharmaceutical composition according to claim 15; and
(b) detecting the radiolabeled compound when associated with MAGL.

17. Use of a radiolabeled compound according to any one of claims 1-12 for diagnostic imaging of monoacylglycerol lipase (MAGL) in the brain of a mammal.

18. Use of a radiolabeled compound according to any one of claims 1-12 for the preparation of a medicament for the diagnostic imaging of monoacylglycerol lipase (MAGL) in the brain of a mammal.

19. The invention as hereinbefore described.
